(19) United States
(12) Patent Application Publication
(10) Pub. No.: US 2022/0313233 A1
(43) Pub. Date: Oct. 6, 2022

(54) MEDICAL IMPLANT, DELIVERY DEVICE, METHOD OF PRODUCING A MEDICAL IMPLANT, AND METHOD OF DELIVERING A MEDICAL IMPLANT

(71) Applicant: HOLISTICK MEDICAL, Paris (FR)

(72) Inventors: Ellen ROCHE, Galway (IE); Matthew KEILLOR, Paris (FR); Philippe POULETTY, Paris (FR); Antoine PAU, Paris (FR); Marco GARD, Borgomasino (IT); Boris WARNACK, Oberwil (CH); Maëlle BRUNEAU, Paris (FR); Tony WEISS, Randwick, NSW (AU); Jason Alan BURDICK, Philadelphia, PA (US); Andrew SPENCER, Boston, MA (US)

(21) Appl. No.: 17/642,263

(22) PCT Filed: Sep. 11, 2020
(86) PCT No.: PCT/EP2020/075550

§ 371 Date: Mar. 11, 2022

(30) Foreign Application Priority Data
Sep. 13, 2019 (IB) PCT/IB2019/001058

(51) Int. Cl. A61B 17/00

(52) U.S. Cl. CPC A61B 17/00491; A61B 2017/00623; A61B 2017/00606; A61B 2017/0065; A61B 2017/00004; A61B 2017/00243

(57) ABSTRACT

The invention relates to a medical implant (1) that is adapted to repair or close defect (D), in particular an opening in a ventricular, atrial, or septal wall (W). The medical implant (1) may, in particular, be a patch. It comprises an adhesive composition (6). It further comprises two states, wherein in the first state, the medical implant (1) can be deployed to an implant site while the adhesive composition (6) is inactive. It can be brought into a second state by an activation mechanism. The adhesive composition (6), in the second state, is curable by a curing mechanism.

The present invention relates to a medical implant, in particular a patch, a delivery device for a medical implant, and a method of delivering a medical implant according to the preamble of the independent claims.

Defects in tissue, for example atrial septal defects (ASDs) or ventricular septal defects (VSDs), are fairly common conditions in humans that are typically treated minimally invasively or surgically. Such defects can cause a variety of symptoms such as shortness of breath and a higher burden on the heart and lungs.

As a consequence, a myriad of implantable devices has been proposed in the prior art, many of which can be deployed in a minimally invasive way.

For example, closure of an atrial septal defect (ASD) by deploying umbrella-like implants through a catheter have been disclosed by Lock et al. (DOI: 10.1161/01.CIR.79.5.1091).

However, known implants exhibit several disadvantages. Typically, they are attached to a tissue wall, or held in place, mechanically. On one hand, this can lead to small injuries of the tissue to be treated. On the other hand, mechanical attachment places constraints on the material choice and mechanical strength of the used components. Finally, mechanical anchoring can also be difficult to realize in a minimally invasive treatment.

Thus, the object of the present invention is to overcome the drawbacks of the prior art, in particular to provide a medical implant, a device for delivering an implant, and a method of delivering an implant that is easy and safe to use.

This and other objects are achieved by the medical implant, the delivery device and the methods according to the characterizing portion of the independent claims of the invention.

The medical implant according to the invention is adapted to repair or close a defect, in particular an opening in a ventricular, atrial, or septal wall. In particular, the medical implant may be a patch, for example a polymeric or pericardial patch. It comprises an adhesive composition. It further comprises two states, wherein in the first state, the medical implant can be deployed to an implant site while the adhesive composition is inactive. It can be brought into a second state, preferably at the implant site, by an activation mechanism. The adhesive composition, in the second state, is curable by a curing mechanism. In particular, it is conceivable that the activation mechanism is identical to the curing mechanism. Alternatively, two different mechanisms may be employed for activation and curing.

The medical implant may also be suitable to close a cavity, such as a left atrial appendage. In particular, the implant may be sized and shaped such that the implant may be attached an ostium of a cavity.

The curing mechanism may in particular be exposure to electromagnetic radiation, for example exposure to visible light, UV light, IR light, and/or X-rays. Curing may, in particular, include cross-linking of the adhesive.

A patch shall be understood as a substantially flat structure. Preferably, it is mechanically flexible such that it can adapt to an underlying surface shape or structure.

The implant is preferably adapted in size and shape to close an opening in a ventricular and atrial septal wall, for example a patent foramen ovale (PFO). Typically, a medical implant for such an application has a substantially round, preferably circular, shape, though any shape such as a triangle, square, or more complicated shape is possible. It is substantially flat, and has a typical diameter of 20-30 mm, preferably 20-25 mm. It may of course be larger or smaller, depending on a patient or the opening to be treated. For example, an opening in the heart of a child may be smaller, requiring a patch diameter as small as 10 mm. It may also be as large as 30 mm, for example if a patient is very tall. Typically, the thickness of the medical implant is 100-200 μm. Of course, the thickness may also be adapted and be as thin as 50 μm or as thick as 1.5 mm, preferably as thick as 1 mm, particularly preferably as thick as 500 μm.

Preferably, the medical implant comprises a material with self-healing and/or self-closing properties. Such properties enable easier implantation because the implant can be temporarily held with an instrument through a hole, for example with a needle and/or a suture, wherein the hole closes automatically after implantation.

Additionally or alternatively, the implant may comprise a hole in a center region that enables or assists holding with a delivery instrument.

In general, materials with high flexibility are preferred materials for the implant according to the invention. A high flexibility reduces the risk of tear, rupture and/or dislodgment during and after implantation. However, it will be understood that rigid materials, in principle, are also suitable for the implants disclosed herein.

Preferably, in the first state, the medical implant comprises at least one cavity that contains the adhesive composition. Preferably, the medical implant comprises a plurality of cavities. It may be adapted to, in the second state, release the adhesive composition.

A cavity shall be understood as a closed structure in the medical implant that can retain another substance such as a liquid, a viscous liquid, or even a solid. For example, the cavity may be a large hollow structure or a small pore. Cavities are particularly advantageous to store the adhesive composition in the first state because the adhesive is protected from the surrounding media, in particular humidity/moisture from the body, for example originating from as bodily fluids. Thus, it does not accidentally engage tissue before activation, and prevents problem while deploying the medical implant, such as clogging of a catheter due to adhesive leaks.

In general, the adhesive may also be electrically activatable. An example of an electrically activatable glue is voltaglue, for example as disclosed in ACS Appl. Bio Mater. 2019, 2, 6, 2633-2642, which is incorporated here by reference. However, any other electrically activatable glue is suitable. In general, such glues may contain an element or molecule that can form a radical when exposed to a voltage. The formed radicals may cause cross-linking.

Preferably, the implant is manufactured by an additive manufacturing/3D-printing method.

Preferably, the implant comprises optical fibers for distribution of light within the implant. For example, the optical fibers may comprise or consist of glass, a polymer, or particularly preferably a biodegradable polymer.

The cavities may have at least two boundary surfaces. At least one property of the boundary surfaces is different between one boundary surface and the other. Preferably, the property includes at least one of a permeability and solubility.

For example, the boundary surfaces may differ in the permeability for an adhesive such as to preferentially release the adhesive at a particular location or side of the cavity. Additionally or alternatively, the permeability for blood or another bodily fluid may differ.

Additionally or alternatively, the boundary surfaces may differ in their solubility in blood or another bodily fluid. Such a difference in solubility may cause preferential release of an adhesive at a particular location or side of the cavity.

In particular, at least one of the boundary surfaces may comprise, preferably consist of, at least one of PEG, PLA, PET, PUs.

The two different surfaces may also be configured such that one surface is adapted to provide adhesion to tissue, while the other surface is adapted to enhance tissue and/or cell growth.

The medical implant may comprise a radio-opaque element. A radio-opaque element may be any element that provides contrast in radio-imaging. Preferably, the radio-opaque element comprises, in particular consists of, barium sulphate, platinum, iridium, and/or tungsten. The radio-opaque element may, in particular, be a wire, a particle, or another marker.

Preferably, the medical implant comprises a support structure, wherein the radio-opaque element is arranged within or formed by the support structure. For example, the medical implant may comprise a backbone made of a polymer that comprises at least one, preferably a plurality of, barium sulphate particles. Alternatively, a similar backbone may be made of a metal that is radio-opaque.

Particularly preferably, a plurality of radio-opaque elements may be arranged in a particular pattern, spacing, geometry or alignment such that data on the relative positions of the radio-opaque elements, for example their spacing and/or alignment determined from imaging data, may provide information about proper adhesion and/or positioning of the medical implant. For example, three markers may be equally spaced around the circumference of the implant. Additionally or alternatively, the radio-opaque element may have a particular shape that provides information on adhesion and/or position of the implant. For example, the radio-opaque element may have a bent shape, which is kept straight by the adhesive force when the implant is attached to a straight surface. Thus, if a bent shape is determined via radio imaging, it would indicate that the medical device is detaching from the tissue. In particular, the radio-opaque element may be adapted such as to not exert a force that is sufficient to dislodge the implant.

Preferably, the radio-opaque element is arranged within or formed by the adhesive composition. For example, barium sulphate particles may be dispersed in the adhesive composition. Additionally or alternatively, the adhesive may comprise a coordination polymer comprising barium ions. Additionally or alternatively, the radio-opaque element may comprise or consist of barium sulphate, iodine, tantalum, iridium, and/or iohexol, which may also be dispersed in the adhesive.

Preferably, the radio-opaque element comprises at least one of barium sulphate and iodine. Iodine is particularly advantageous if the radio-opaque element is used to track the degradation of an adhesive, a patch material, or other parts of the implant. For example, if the medical implant is designed to degrade in the human body while allowing for cell overgrowth, iodine may be incorporated in a biodegradable material. The degradation of the biodegradable material may be tracked by means of radio imaging. If, for example, coagulation suppressant therapy is needed during degradation, but only during degradation, the imaging data may indicate whether coagulation suppressant are still needed.

Preferably, the implant comprises at least one discrete marker. Discrete shall be understood as contained to a particular area or location. For example, a discrete marker may be used to designate one side of the implant from the other, or to designate an upper and a lower part. Additionally or alternatively, the discrete marker may be deformable by pressure and indicate a pressure at a particular location of the implant, for example a pressure caused by the adhesive force between a tissue wall and the implant. Particularly preferably, the discrete marker may be a spring that may cause detachment if the adhesion force between the implant and the tissue is below a certain threshold and thus facilitates detection of detachment (similar to predetermined breaking point).

The marker may, in particular, be used to guide a robot, preferably a microrobot, to the implantation site after the implant has been implanted. The marker may define a position in the body and may in particular retain its function for a certain period of time, for example one year. Thus, guiding of a robot may also be done a certain amount after implantation. The marker may be detectable by the robot and thus enable passive guiding. Alternatively, the marker may emit a signal that is detected by the robot and thus enable active guiding.

Preferably, the discrete marker is at least one of radio-opaque and echo-opaque/echogenic. Particularly preferably, the discrete marker is configured as being the radio-opaque element.

The implant may comprise at least two discrete markers that are arranged at a pre-defined distance and/or orientation from one another.

Preferably, the medical implant has a generally flat shape with a first and a second surface, wherein the first and the second surfaces are facing in substantially opposite directions. At least one property of the first surface is different from a corresponding property of the second surface.

The person skilled in the art will understand that “generally flat” may encompass slightly curved flat surfaces and shapes, in particular disk- or chip-like shapes.

The difference between the first and the second surface may be with respect to any measurable quantity, wherein a measurement of said quantity would yield significantly different values. Particularly preferably, the first and second surface differ in polarity, charge, functionalization, surface structure, surface pattern, material, coating, and/or porosity.

The first surface may be adapted to enhance cell ingrowth. In particular, the porosity of the surface may be adapted to allow for cell ingrowth, for example by having a pore size of adapted to allow for cell ingrowth. The pore size may be in the range of a few microns to several hundred microns. Preferably, the pores have a diameter between 50 μm and 500 μm. Additionally or alternatively, the first surface may be biocompatible and in particular be functionalized with growth factors or cell adhesion motifs. The first surface may comprise surface charges that activate and/or attract cells. The first surface may also have a surface roughness adapted to enhance cell ingrowth and/or comprise a velour-like surface.

In particular, at least one of a length, size, and 3D-arrangement of pores and holes may be adapted to enhance cell ingrowth. The length may in particular denominate the longest extension along an axis of a pore or hole in case of nonspherical pores/holes.

Preferably, the first and/or the second surface may comprise or consist of derivates of polymer peptides.

Preferably, the second surface is adapted to provide adhesion to biological tissue. In particular, the biological tissue may be human or animal tissue such as one of endocardial, pericardial and septal tissue. For example, the second surface may comprise a glue layer.

Preferably, at least one surface of the implant, in particular at least one of the first and the second surface of the implant, comprise a velour-like surface. Particularly preferably, all surfaces of the implant comprise a velour-like surface.

Preferably, the adhesive composition is arranged on the medical implant in a pattern. The pattern may be non-uniform. In particular, the pattern may be printed on the implant by inkjet or extrusion printing. The pattern may also be regular, but comprise a 3D structure and/or a non-homogeneous topography.

Preferably, the adhesive composition comprises gelatin-methacryloyl (GelMA), in particular a GelMA of animal original. Particularly suited GelMAs are Fish GelMA, porcine GelMA, and bovine GelMA, i.e. GelMA processed and originating from fish and/or pigs and/or cows. GelMA originating from cold-water fish is particularly suited because of its low-temperature (in particular at room temperature) mechanical flexibility. However, any type of commercially available GelMA is suitable for the invention.

In particular, the GelMA, in particular if derived from pigs, may have a Bloom value of 250 to 325. GelMA derived from fish may not have bloom strength.

Preferably, the GelMA has a molecular weight of 50 to 170 kDa.

Preferably, the GelMA may be formed by a mixture of at least two GelMAs of animal origin. Particularly preferably, the GelMA is formed by a mixture fish GelMA and porcine GelMA.

A mixture of two GelMAs enables to combine properties of different GelMAs. For example, a mixture of (cold water) fish GelMA with porcine GelMA may yield a GelMA with the solubility of porcine GelMA and the mechanical flexibility of fish GelMA. It is also possible to gradually tune properties, for example solubility and mechanical flexibility, by choosing the ratio of different GelMAs, for example porcine and fish GelMA. Such GelMAs are known to the skilled person and commercially available.

Additionally or alternatively, it is also possible to tune properties of the GelMA by varying and/or mixing of different molecular masses.

Preferably, the adhesive composition further comprises at least one photoinitiator. The adhesive composition may comprise several different photoinitiators, a single photoinitiator, or mixtures of different photoinitiators.

The photoinitiator may be a so-called type I photoinitiator, preferably one of lithium phenyl-2,4,6-trimethylbenzoylphosphinate, LAP, Irgacure, and camphorquinone.

Additionally or alternatively, the photoinitiator may be a so-called type II photoinitiator, preferably one of Eosin Y and mono/di/triethanolamine, Rose Bengal and mono/di/triethanolamine, and Riboflavin and mono/di/triethanolamine.

The GelMA may also be cross-linkable by X-ray radiation. Particularly preferably, the GelMA is cross-linkable via a photoinitiator that can be activated by X-rays. Alternatively, the GelMA may be crosslinkable without a photoinitiator.

The medical implant may also comprise a rivet, in particular a blind rivet. A rivet may be used for attachment of the implant to the tissue.

Rivets are particularly advantageous because they are not sensitive to, and thus securely attach in the presence of, different temperatures, chemical environments, and humidity levels. Preferably, a rivet can thus be used to at least temporarily attach the medical implant to tissue, for example during curing of the adhesive composition, during placement of the implant, and/or while other manipulations on the implant are performed by a medical professional.

The medical implant may comprise at least one retaining element for retaining at least one suture at an outer circumference of the implant. In addition, a suture can be arranged within said loop.

Such a retaining element provides a particularly easy method of temporarily attaching the medical implant to a delivery device. In particular, the suture(s) may provide a connection to the delivery device. Tearing of the suture(s) and/or the retaining element can release the implant.

Preferably, the retaining element comprises or consists of a loop of fabric. Alternatively, the retaining element may also be formed by a backbone folded such as to form a loop in a circumferential area of the implant.

The retaining element may also comprise a pre-determined breaking point.

Preferably, the at least one retaining element is formed from the same material as the medical implant.

Additionally or alternatively, the at least one retaining element is formed of polyurethane with a thickness of 35 to 65 μm. Preferably, the thickness is between 45 and 55 μm.

Alternatively, the at least one retaining element is configured as a separate element arranged on the medical implant. For example, the retaining element may be configured as a separate strip of fabric attached to a circumferential area of the implant and folded such as to form a loop. Alternatively, the retaining element may comprise a loop formed by a suture.

Preferably, the at least one retaining element comprises a predetermined breaking point, particularly preferably an indentation. This allows to control where the retaining element breaks when releasing the implant and as such provides better control the implant procedure.

The adhesive composition may also comprise, in particular consist of, a dried adhesive composition. The dried adhesive composition may swell and/or become at least partially liquid when exposed to a liquid. The dried adhesive composition may also be cross-linkable by exposure to a liquid, such as cyanoacrylate. For example, GelMA as described herein may be dried and used in this manner. Alternatively, any film-forming polymer, particularly biopolymers, such as hyaluronic acid, collagen, heparin and their photopolymerizable counterparts (such as collagen methacrylate) are suitable as well.

The dried adhesive composition is preferably activatable by exposure to a liquid, for example by rehydration in saline, blood, and/or water. Additionally or alternatively, the dried adhesive may also be activatable by exposure to cyanoacrylate. After rehydration, the previously dried adhesive may be curable by exposure to electromagnetic radiation, such as visible and/or UV light, in the presence of a photoinitiator.

Preferably, in the first state, the medical implant comprises a plurality of cavities, in particular micro-sized cavities.

Micro-sized cavities shall be understood as cavities with any shape that have characteristic size in the micrometer range, i.e. from 1 μm to 1000 μm. For example, the medical implant may comprise a plurality of spherical cavities with a diameter of 10 μm to 100 μm. This is particularly advantageous because it ensures a homogenous distribution, and if necessary mixing, of the adhesive composition when it is released.

Preferably, the at least one cavity comprises an additive that, upon exposure to humidity, swells. The at least one cavity may be adapted to release the adhesive composition upon swelling.

This enables a particularly easy way of releasing the adhesive, because exposure to blood automatically makes the cavities swell and thus releases the adhesive.

Preferably, the medical implant comprises at least two different kinds of cavities.

The two types of cavities may be different in size, composition, shape, or any other property. For example, the two different types of cavities may contain different additives that make them swell at different rates. They may also have different wall thickness, different radii, or be made of a different material. They may also be adapted such that one type of cavity swells and the other does not. This enables better control of the release of the adhesive. For example, one component can selectively be released first, or one component can be released at a different rate than the other. It may also enable a better control of the rate of release if the adhesive only comprises one component. For example, it may be advantageous to release a first fraction of the adhesive, and release a second fraction at a later point. It is also possible to adapt the cavities such that they release the adhesive composition or a component thereof at different pressures or temperatures.

Preferably, the at least two different types of cavities are adapted to contain different components of an adhesive composition, in particular in a liquid, gel, dried, or gaseous form. This may, in particular, include any of the features of two different types of cavities as described above. However, it may also include particular properties that enable the storage of a particular component of an adhesive. For example, a certain wall material may be particularly advantageous for one component of an adhesive composition, but may be incompatible with another. Thus, it may be advantageous to adapt the cavities to the specific components of an adhesive composition. Of course, it may also include different sizes or degradation rates of the cavities to account for desired ratios of two components in the final (mixed) adhesive composition.

Preferably, the adhesive composition comprises two components that are individually disposed in the cavities, such that in the first state, the two components are separated. This enables the controlled mixing in the second state, for example at the implant site. This is of course particularly advantageous for two-component adhesives that are not curable before mixing. In this case, unplanned curing before adhesive release, for example by accidental exposure to humidity or elevated temperature can be prevented. However, it is of course conceivable to have an adhesive that comprises one component that only additionally hardens the adhesive composition, for example through cross-linking. It may be advantageous to dispose such a component separately as well.

Preferably, the adhesive composition is adapted to be curable upon mixing of the at least two components. This prevents accidental curing before release of the adhesive. Any curing mechanism to cure the curable adhesive composition after mixing is conceivable. It may be an increase in temperature, exposure to humidity, exposure to electromagnetic radiation such as visible light, infrared light, ultraviolet light, or a combination thereof.

Preferably, the adhesive composition is adapted to spontaneously cure upon mixing of the at least two components. This offers a particularly advantageous way of deploying the adhesive composition because it does not require additional processing steps beyond the release and the spontaneous mixing of the two components. For example, a first component may comprise a primary amine, and a second component may comprise an NHS ester. In the presence of the functional groups of the first and second components, the adhesive may cure upon mixing. However, it is of course possible to combine such an adhesive composition with additional curing mechanisms. For example, an adhesive composition with two components may spontaneously cure upon mixing, but exposure to an additional curing mechanism such as electromagnetic radiation, humidity, or increased temperature may accelerate the curing if necessary. It is also conceivable that a third component is used as an additional hardener.

Preferably, the cavities are adapted to release the adhesive composition upon a temperature increase, in particular to the temperature of a human body. This enables the automatic release of the adhesive composition after implantation because the implant is heated up to 37° C.

Preferably, the cavities are adapted to release the adhesive composition upon exposure to electromagnetic radiation. For example, the cavities may degrade or burst upon irradiation with visible light, infrared light, ultraviolet light, or similar. They may also be adapted to release the adhesive composition upon irradiation with a particular wavelength or wavelength range. If more than one type of cavity is present, they may also be adapted to release the adhesive upon irradiation with different wavelength ranges, such that it is possible to selectively release one component at a time. In general, cavities that are adapted to release the adhesive composition upon exposure to electromagnetic radiation are particularly advantageous because they enable the combination with a delivery device that can transmit light for the release, for example delivery devices as disclosed in WO 15/1756632. It is also a particularly safe way of releasing the adhesive composition because light is typically not prevalent in the human body, thus preventing accidental release.

Preferably, the cavities are adapted to release the adhesive composition upon an increase in pressure. In particular, this enables the release of the adhesive composition upon inflation of a balloon. However, any other mechanism to provide a pressure increase is also conceivable. For example, the cavities may be adapted such that exposure to a liquid causes swelling due to an osmotic pressure that provides a pressure difference. It may also comprise a separate inflation reservoir to provide a pressure on the cavities, the implant, or another part thereof.

Preferably, the cavities are adapted to release the adhesive composition upon a mechanical compression. For example, a compression by a balloon of a delivery device may be employed and is particularly advantageous because delivery devices with balloons are known and thus easy to implement.

Preferably, the adhesive composition is adapted to be curable by exposure to electromagnetic radiation. This provides the similar advantages already described in the context of cavities that are adapted to release upon exposure to electromagnetic radiation. For example, the adhesive composition may be curable upon irradiation with visible light, infrared light, ultraviolet light, or similar. It may also be adapted to be curable upon irradiation with a particular wavelength or wavelength range. In general, adhesive compositions that are adapted to release the adhesive composition upon exposure to electromagnetic radiation are particularly advantageous because they enable the combination with a delivery device that can transmit light for the release, for example delivery devices as disclosed in WO 15/1756632. It is also a particularly safe way of curing the adhesive composition because light is typically not prevalent in the human body, thus preventing accidental curing.

Preferably, the medical implant comprises a self-expanding support structure. A support structure may be any structure with a higher mechanical stiffness and/or strength than the rest of the implant. For example, it may comprise at least one strut of a polymeric material that provides mechanical stiffness to the implant. Additionally or alternatively, it may also comprise a structure that holds the medical implant in place at a desired implant location such as a PFO. For example, it may comprise structure that is adapted to be placed in a defect and hold two patches—one on each side of the defect. Alternatively, it may also only hold one patch. The self-expanding property of the support structure enables particularly easy deployment through a catheter. For example, it may be made of a shape memory material such as a shape memory polymer or a shape memory metal such as a Nitinol. It is also conceivable, however, to use an elastic material that is compressed in a delivery device and expands into its original shape at the implant site.

Preferably, the cavities are formed by closed capsules. In particular, the cavities may be formed by spherical capsules. They may be adapted to be opened by the activation mechanism.

Preferably, the capsules are adapted to break open by the activation mechanism. They may be adapted to break open by any of the activations described herein, for example exposure to electromagnetic radiation, increased pressure, increased temperature, or a combination thereof.

Preferably, the capsule walls are adapted to dissolve by the activation mechanism. This is particularly advantageous if they are adapted to dissolve upon exposure to water, blood, or another bodily fluid, because such an activation mechanism does not require additional handling or processing due the natural presence of these fluids at the implant site. Thus, this provides a particularly easy and safe way of release the adhesive composition from the capsules.

Preferably, the capsule, in particular the capsule wall, comprises a filler material. Filler materials can increase the mechanical strength of the cured adhesive. Thus, such capsules can serve a double purpose by holding the adhesive composition and upon release provide a filler material.

Preferably, the medical implant comprises a porous foam and, in the first state, an adhesive composition is disposed in the porous foam. Of course, it is possible to combine a foam with other cavities as described herein. For example, the medical implant may comprise cavities that are larger than the pores of the foam and contain a second component of the adhesive, a hardener, or another substance. A foam shall, in particular, be understood as a material that substantially consists of pores separated by pore walls made of a solid or a liquid component. The pores of a foam typically exhibit sizes and shapes that fluctuate statistically. Typically, they also percolate.

In a particularly preferred embodiment, the medical implant comprises a braided structure that supports the porous foam.

Preferably, the porous foam comprises solid walls.

Preferably the porous foam comprises walls formed of a hydrogel. Hydrogels are particularly advantageous because they are typically biocompatible. In addition, they can be manufactured from a wide variety of different materials and can thus be adapted to the application or even the patient to be treated. In addition, hydrogels can functionalize and comprise active components. Hydrogels can also be adapted to biodegradable.

Preferably, the pore size of the foam is adapted to allow for cell ingrowth. This facilitates the formation of tissue in or around the foam. In particular, this is advantageous in combination with biodegradable materials such as a hydrogel that is adapted in this way. The medical implant can then be adapted to degrade at a rate that is slower than cell ingrowth. Thus, the medical implant can serve as a template or scaffold and be degraded in the body once the implant is replaced with body tissue.

Preferably, the medical implant comprises at least one reservoir, and at least part of the adhesive composition is disposed in said reservoir. A reservoir shall be understood as a compartment in the medical implant with a characteristic size in the order of magnitude of the implant itself. It may be disposed as a cavity in the medical implant. However, it may also be arranged as a separate reservoir that is attached to the implant. For example, it may be a blister on a surface of the medical implant. A reservoir can be advantageous if a relatively large amount of adhesive needs to be released fast, and/or it is only needed at a specific location.

Preferably, the medical implant comprises at least one separate inflation reservoir. This allows for localized inflation and thus release of an adhesive disposed in a cavity or reservoir. For example, it is possible to inflate a reservoir in a particular location of the medical implant first, releasing a first portion of the adhesive. The rest of the adhesive can then be released at a second point in time. Similarly, it would be conceivable to arrange two inflation reservoirs to selectively release two portions at, for example, two different locations separately.

In a particularly preferred embodiment, the at least one reservoir filled with adhesive is adapted to release said adhesive upon inflation of the at least one inflation reservoir. The medical implant thus comprises at least one reservoir which is at least partially filled with adhesive and at least one inflation reservoir. However, it is of course also possible for the medical implant to comprise several inflation reservoirs and/or several reservoirs filled with adhesive. In particular, one inflation reservoir may be used to release the adhesive from more than one reservoir filled with adhesive. Similarly, two or more inflation reservoirs may be adapted to release the adhesive from one reservoir filled with adhesive. This can provide additional safety through redundancy, or can also be used to provide an easy way to release a defined first and second portion of the adhesive from one reservoir.

Preferably, the reservoir is adapted such that the adhesive is only released on one side of the medical implant. In particular, it may only be released on a distal side of the patch, or on a proximal side of the patch. However, it is also conceivable that the adhesive would be released only on a side wall side of the medical implant. This ensures proper placement of the adhesive facing the tissue and prevents release of adhesive where the medical does not and is not designed to be in operative contact with tissue. As a consequence, the necessary amount of adhesive composition is also reduced. This is more economical and safer for the patient. The medical implant may adapted to only release the adhesive on one side by selecting different materials on each side, by varying the thicknesses and/or the density of the material on each side, by varying the pores size and/or structure of a foam, or by changing any other property that may change the permeability of the implant material for an adhesive composition.

Preferably, the medical implant comprises at least one microchannel in fluid connection with the reservoir for the release of the adhesive. A microchannel shall be understood as a channel with a longitudinal shape that is in fluid connection to a surface of the medical implant and has a diameter that is small compared to the size of the medical implant and the reservoir.

In particular it may have a channel diameter in the range of 1 to 1000 μm, preferably 25 to 750 μm, even more preferably 50-300 μm. Such microchannels enable an easy release of the adhesive composition from the reservoir.

In a particularly preferred embodiment, the medical implant is adapted to only release the adhesive composition on one side of the implant and comprises at least one microchannel. In particular, the implant may be adapted such that the adhesive is only released through said microchannel. Thus, the arrangement of the microchannels provides an easy way of adapting the medical implant to release the adhesive in a particular location, for example only on one side of the implant.

Preferably, the adhesive composition is disposed as fibers, in particular solid fibers. Even more preferably, the adhesive composition comprises at least two components, at least one of which is disposed as solid fibers. Solid fibers may comprise a dried adhesive, or an adhesive that can be molten (such as a hot melt), or an adhesive that swells if exposed to humidity or water. In particular, an adhesive composition may also be disposed as a coating on a fiber.

In particular, at least one of the fibers can comprise an aldehyde that is adapted to adhere to tissue.

[30] Preferably, the adhesive composition is adapted to at least partially dissolve upon exposure to a liquid, in particular one of an organic solvent, a saline, and blood, and form a gel, in particular a hydrogel.

Preferably, the adhesive composition comprises at least one component from the group of methacrylated gelatin, methacryloyl-substituted tropoelastin, poly(acrylic) acid, and methacrylated collagen. Poly(acrylic) acid may be used with a diacrylate/dimethacrylate/amide crosslinker

Preferably, the adhesive composition comprises a dried component that is adapted to be curable by a curing mechanism upon exposure to a liquid, in particular an organic solvent or blood. This is particularly advantageous because it allows for an automatic activation of the adhesive upon implantation due to the presence of blood. Of course, it is also possible to adapt the adhesive composition such that only a selective activation by an organic solvent is possible. It may also be adapted such that exposure to blood activates the adhesive, but an additional exposure to an organic solvent accelerates the activation process.

In particular, it is also possible to adapt the adhesive such that it can be activated by a liquid either inside the body of outside the body before implantation.

In particular, the adhesive composition may be activatable by rinsing with a solution comprising a photoinitiator. Alternatively, the photoinitiator may also be comprised in a dried adhesive and become activated by exposure to a liquid. For example, it may only be reactive in an at least partially swollen adhesive and be kinetically hindered in the dried solid adhesive.

Preferably, the medical implant comprises a scaffold made of bioabsorbable or biodegradable material (hereinafter, reference to “biodegradable” materials shall be understood as encompassing both, bioabsorbable and biodegradable materials), in particular a woven, knitted, electrospun, melt spun, and/or nonwoven bioabsorbable material, and/or a biological implant made by 3-D printing. A scaffold may in particular be used to facilitate cell in-growth and tissue formation.

The invention also relates to a method for deploying a medical implant that comprises and adhesive. The method is particularly advantageous in combination with a medical implant as described herein, but of course it is possible to perform the method with any other medical implant that comprises an adhesive. The method comprises the steps of deploying the medical implant to a first site in a first state, bringing the medical implant into a second state by means of an activation mechanism, and curing the adhesive by means of a curing mechanism. The first site is preferably the implant site. However, it is also possible to bring the implant into the second state either outside the body or inside the body, but not at the implant site.

Preferably, the method comprises a step of increasing the temperature of the medical implant to bring it into the second state. In particular, an increased temperature may be used to release an adhesive as described herein, for example by breaking open capsules and/or dissolving cavities.

Preferably, the temperature increase is at least partially provided by and external heat source. It may also be provided only by an external heat source.

Alternatively or additionally, the temperature increase is at least partially provided by a patient's body heat. It may also be provided only by the patient's body heat.

Alternatively or additionally, the temperature increase is at least partially provided by electromagnetic radiation, in particular infrared light. It may also be provided only by electromagnetic radiation.

Preferably, the method comprises a step of applying a pressure to bring the implant into the second state. For example, the applied pressure may squeeze the adhesive composition out of capsules or cavities. It may also release the adhesive composition from the pores of a foam.

Preferably, the pressure increase is at least partially caused by osmotic pressure. It may also be only caused by osmotic pressure. For example, the adhesive composition may comprise a concentration of ions that is higher than the one of blood and may be comprised in a plurality of cavities with a wall through which water can diffuse. Due to osmotic pressure, water diffuses into the cavities. The cavities can be adapted such that they burst at pressures lower than the osmotic pressure. Of course, the same can be achieved with only one or any other number of cavities.

Preferably, the pressure increase is at least partially caused by applying mechanical deformation, in particular a mechanical pressure, to the implant. The person skilled in the art will of course understand that all the possibilities of increasing the pressure described herein can be combined.

Preferably, the method comprises the step of exposing the implant to humidity to bring it into the second state. Humidity may, for example, cause the dissolution or bursting of cavities or capsules. It may also cause swelling of an adhesive composition to bring it into a curable or cured state. Any other mechanism of releasing the adhesive and/or bringing into a curable state described herein is conceivable.

Preferably, the method comprises the step of exposing the medical implant to electromagnetic radiation to bring into the second state. The electromagnetic radiation may be infrared light, ultraviolet light, or visible light. It may cause degradation of cavities or any other mechanism described herein to release and adhesive from a cavity and/or bring it into a curable state.

Preferably, the method comprises the step of spontaneous mixing of two components in the second state, which causes curing of the adhesive. For example, the adhesive composition may comprise two components that are released from separate capsules by any of the mechanisms herein. Upon release, the mix spontaneously mixes due to the loss of the separation in the cavities. The adhesive composition may thus be adapted such that the two components react with each other without any other external trigger. This provides a particularly easy way of curing the adhesive composition. However, it would of course be possible to combine such an adhesive composition with an additional curing mechanism to cure or harden it further.

Preferably, the method comprises the step of exposing the medical implant to electromagnetic radiation in the second state, which causes curing of the adhesive.

Preferably, the method comprises the step of inflating a separate inflation reservoir to bring the implant into the second state. In particular, inflating of the separate inflation reservoir may squeeze the adhesive out of a reservoir containing the adhesive.

Preferably, the method comprises the step of disposing a liquid to bring the implant into the second state. The liquid may be an organic solvent, in particular an organic solvent that is miscible with human blood. The liquid may, however, also comprise or even consist of water, in particular a physiological saline solution.

The invention is further directed at a medical implant. The medical implant comprises at least one tear line that is arranged in proximity to a circumference of a medical implant. The medical implant is adapted to repair or close a defect, preferably an opening in a ventricular or atrial or vessel wall. In particular, the medical implant may be a patch or any other implant as disclosed herein. The medical implant is further adapted such that, upon deployment to an implant site, in particular a septal defect site, the medical implant can be attached to a tissue wall and torn along the tear line.

A tear line shall be understood as any sort of weakening in the material that creates a site of predetermined failure. Thus, if a mechanical stress is applied to the material, the material will first break along the tear line. This allows for an attachment of the medical implant to a delivery device and easy release from the delivery device by breaking or tearing along the tear line. The tear line may, for example, be a weakening of the material by aligned holes or perforations in the material, or be a lower thickness along a certain line, or a region of a different material.

The tear line may be arranged around a complete circumference of the medical implant, or only at selected regions where the implant is attached to a delivery device. In particular, the medical implant may also comprise elongated elements for attachment to a delivery device and that are detachable from the medical implant by means of a tear line. For example, such elongated elements may be flaps or arms of the same material as the implant, or struts of another material such as a polymer or a metal.

A preferred way of tearing the tear line is by inflation of a balloon on a delivery device. Thus, the tear line is preferably arranged such that a mechanical stress can be applied to it by means of a balloon. For example, the medical implant may comprise tearable flaps that are too short to reach around the circumference of a balloon. Thus, the tear lines would be arranged perpendicularly to the longitudinal axis of the flaps. Inflation of the balloon causes a mechanical stress along the longitudinal axis of the flaps and thus rupture along the tear line.

Preferably, the medical implant comprises a biodegradable material that is adapted to lose its mechanical strength in a human body within three years, preferably twelve months, even more preferably six months. Loss of mechanical strength shall in particular encompass molecular weight loss of a polymeric substance which reduces the mechanical stiffness.

Preferably, the medical implant is coated with a non-adhering coating, in particular silicone and/or poly(tetrafluoro ethylene), between an outer edge and the tear line. This prevents adhesion of the medical implant to tissue in areas that are designed to be retracted with the delivery device.

Preferably, the medical implant comprises a cut, in particular a cross-shaped cut, that is adapted such that a delivery device can partially extend through said cut. The cut may have any shape that provides an opening in the medical implant that can be closed. For example, it may also be a semi-circular cut that forms similarly shaped flap, or a square-shaped cut. Even a linear cut is conceivable, but would have to be long enough for a delivery device to extend through it. In addition, the cut is adapted such that the opening formed by it at least has a tendency to close itself. This allows for the delivery device to at least partially extend on both sides of the implant during delivery, but to be retracted through the medical implant after delivery. After retraction, the flaps close the opening.

Preferably, the medical implant comprises fibers, in particular woven, spun, or knitted fibers. This is particularly advantageous if the medical implant comprises, or consists, of a fabric patch. The fibers can be adapted for certain functions, such as being coated with an adhesive or a drug and/or being biodegradable. Of course, it is possible to combine different types of fibers in one medical implant, for example fibers coated with different adhesive, adapted to biodegrade at different rates, or to elute different drug, or any combination of those.

Preferably, the fibers are made of a biodegradable material.

Preferably, the biodegradable material is selected from a group of poly(lactic-co-glycolic acid), poly(L-lactic acid, poly(D-lactic acid), poly(glycolic acid), poly(caprolactone), any copolymer and/or blend of these materials. These materials are particularly advantageous in that they are non-toxic, well-known and approved for medical applications, and easily commercially available.

Preferably, the medical implant comprises a spine structure. In particular, the spine structure may have different mechanical properties from the rest of the medical implant. For example, it may be made from a different material, or have different dimension such as a greater thickness. The spine structure allows for tuning of the implant properties with more flexibility because the mechanical properties can be tuned without necessarily changing the implant material that may have been selected due to other properties.

Preferably, the spine structure comprises, particularly preferably consists of, a polyurethane.

Preferably, the spine structure extends beyond the medical implant to provide non-elastic tear arms. The non-elastic tear arms can be used to connect the implant to the delivery device, in particular a center lumen. This enables a more reliable implant, in particular patch, release without constraining the choice of material.

Particularly preferably, the retaining element for retaining at least one suture is formed by or on the non-elastic tear arm.

It is also possible to configure the non-elastic tear arms as an extension of the implant, for example by cutting out an implant including extending flaps from a sheet of implant material.

Preferably, the medical implant comprises an adhesive composition, in particular an adhesive composition that is adapted to attach the medical implant to human tissue. In particular, any adhesive composition as described herein may be used. It may preferably comprise glutaraldehyde for pre-treatment of the tissue to which the medical implant is to be attached. In particular, the adhesive composition may comprise derivates of a polymer with linkers that covalently bind to cell surface molecules. Additionally or alternatively, the adhesive composition may include growth factors, chemotactic factors, coagulation or anticoagulation factors, and/or anti-inflammatory compounds incorporated into it.

Preferably, the tear line comprises a laser-cut tear line. This provides a particularly easy and precise way of manufacturing a medical implant with a tear line.

Preferably, the medical implant comprises at least one extension that radially extends beyond the periphery of the medical implant. This allows for attachment to a delivery device.

In a particularly preferred embodiment, the medical implant comprises at least one extension that radially extends beyond the periphery of the medical implant and a spine structure. In particular, the spine structure may also comprise non-elastic tear arms as described herein, wherein the non-elastic tear arms are arranged such as to be comprised in the extension.

Preferably, the at least one extension comprises a string and/or a suture. In particular, the extension may consist of a string and/or a suture. This is particularly advantageous because it provides a simple way of attaching the medical implant to a delivery device, for example by means of a knot or a suture.

Additionally or alternatively, the at least one extension comprises a strip consisting of the same material as the medical implant. This is particularly easy to manufacture because the medical implant can be, for example, cut from the base material with the extension directly.

The retaining element for retaining at least one suture may be formed by the at least one extension.

Preferably, the at least one tear line is adapted, in particular located and dimensioned, such as to separate the at least one extension from the medical implant upon tearing. If the medical implant comprises a spine structure with non-elastic and/or non-extensible tear arms, the tear line may also be arranged such as to weaken the non-elastic tear arms at the same or a different location as the at least one extension.

Preferably, the extensions are adapted to hold the medical implant, preferably a patch, to a delivery device, in particular balloon comprised in a delivery device.

Preferably, the implant is formed by a part of a surface of an inflatable balloon of a delivery device. The balloon may be made of an implant-grade material such as polyurethane. It may also comprise a tear line that is adapted to break at a predetermined pressure or pull force. In particular, the tear line may be formed by a weakening of the material other than holes or cuts to allow for efficient inflation. For example, a circumference of the balloon may have a thinner wall such that the balloon ruptures along said tear line. The balloon, or an area inside a tear line, may also be coated with an adhesive.

The invention is further directed to a delivery device to deliver a medical implant comprising a tear line as described herein, in particular a medical implant comprising an adhesive. It comprises a shaft with an implant holder for holding the implant.

The implant holder is adapted to hold the implant, preferably at least partially, along its periphery. In particular, it may hold the implant by means of an adhesive ring on the periphery of the medical implant. The delivery device further comprises and actuation mechanism for increasing a distance between at least two predefined points of the implant such that upon actuation of the actuation mechanism, said tear line is at least partially ruptured.

Preferably, the actuation mechanism includes an inflatable balloon.

The delivery device, in particular the implant holder, may have a retention element for a suture. The retention element is adapted to retain at least one suture connected or connectable to the medical implant.

A retention element is in particular adapted be in operable connection, i.e. to hold, implants that comprise a retaining element with a suture.

Preferably, the delivery device comprises a gauge, particularly preferably arranged on a handle, for indicating an adhesion force between the medical implant and a tissue.

The indicated adhesion force may be measured by the delivery device directly, for example be pulling a part of the implant and measuring a force. If the implant detaches, which could be detected by a sudden dislocation, the force could be determined. Alternatively, the applied force could be measured without detaching, which would provide a minimum value for the adhesion force.

Alternatively, it is also possible that the gauge indicates a value determined by a marker, for example a marker which is deformable under pressure, on the implant.

The invention is further directed to a medical implant, preferably a patch, preferably a medical implant as described herein, in particular a medical implant adapted to close a defect, preferably an opening in a heart wall, in particular an atrial, ventricular and/or septal wall, or vessel wall, or any other defect as described herein. The medical implant comprises at least one connecting element, in particular a bead, that is located at the outer edge of the medical implant. The connecting element has a size that is larger than a size, in particular a thickness, of the implant such that the connecting element is adapted to be brought into engagement with a delivery device having an appropriate counter element for a connection with said connecting element.

Preferably, the beads comprise a polymeric or metallic material. In particular, they may entirely consist of a polymeric or metallic material. They may be attached to a wire or a suture.

The invention is further directed at a delivery device for a medical implant, in particular a medical implant comprising a connecting element as described herein. The delivery device comprises at least one tube comprising an actuation element. The actuation element may, in particular, be a wire located within said tube. It further comprises at least one holder, preferably at least two holders, that are in operative connection with said actuation element, in particular attached to said wire. The tube and the at least one holder, preferably the at least two holders, are adapted such that a medical implant is held by the at least one holder in a first state. The medical implant can be released by actuation of the actuation element.

The invention further relates to a method of producing a medical implant, preferably a medical implant as described herein. An adhesive composition is at least partially liquid and is arranged on at least one surface of the medical implant. The adhesive composition is dried.

The adhesive composition may be dried subsequently to its arrangement on the implant, or may be dried first and then arranged on the implant.

In particular, the adhesive composition may be dried under a vacuum, or at least a pressure lower than atmospheric pressure. Additionally or alternatively, an elevated temperature may also be employed for drying the adhesive composition.

The invention further relates to a method of producing a medical implant, preferably a method as described above. The method is preferably used to produce a medical implant as described herein. At least one extension element is arranged at an outer circumference of the medical implant. The extension element preferably comprises a pre-determined breaking point. The at least one extension element is configured to form a retaining element. To this effect, an end of the extension element may in particular be arranged on a surface of the medical implant such that the extension element forms a substantially closed loop. The end of the extension element is bonded such as to fix the extension element in a configuration comprising a retaining element. Preferably, the method further comprises the step of arranging a suture in the retaining element, in particular in the substantially closed loop.

The substantially closed loop may in particular be formed by folding of the extension, for example such as to arrange two ends of the extension in proximity to each other and bonding the two ends together.

Bonding is preferably achieved by application of a glue/adhesive composition. Additionally or alternatively, bonding may also be achieved by welding, soldering, casting, and/or mechanical attachment (rivets, hooks, Velcro, stitching).

Preferably, the adhesive composition is arranged on the medical implant via inkjet or extrusion printing.

In particular, inkjet or extrusion printing enables complex structures and patterns of adhesive to be arranged on medical implants that may otherwise be difficult to achieve due to brittleness of the adhesive composition upon drying.

Alternatively, it is also possible to arrange a continuous film of adhesive, wherein a pattern is created with a stamp while the adhesive is in a liquid state.

Particularly preferably, the adhesive composition is arranged in a pre-defined pattern such as to enable flexibility of the implant in certain directions. For example, the adhesive composition may be arranged as slices of a round disk. The implant may then be flexible along the axes separating the individual slices. Additionally or alternatively, the pre-defined pattern may include spikes, pyramids, triangles, cubes, barbs, quills, or other shapes.

The pre-defined pattern may be a two-dimensional pattern, i.e. a substantially flat adhesive film with a patterned structure. Alternatively, the pattern may also be three-dimensional, i.e. also comprise a pattern along an axis perpendicular to the implant surface on which the adhesive composition is arranged.

A three-dimensional pattern is particularly advantageous as it allows for local tuning of pressure. For example, a pyramid that extends from the surface may be pressed against tissue with a higher local pressure than a flat film. Such structures may thus also enhance tissue integration through diffusion into the tissue.

The invention further relates to a method of treating a defect, in particular an opening in a ventricular or atrial or vessel wall. The method comprises a step of implanting a medical device, preferably a medical device as described herein. The implant comprises an adhesive composition. The adhesive composition may be hydrated in situ. Alternatively, the adhesive composition may be hydrated prior to delivery by flushing.

Hydration may be passive, i.e. via liquid water and/or vapour that is naturally present in blood or other bodily fluids, or may be active, i.e. via delivery of a liquid, for example through a fluid canal in the delivery device.

The invention further relates to a method of treating a defect, in particular an opening in a ventricular, atrial, septal, or vessel wall. The implant is preferably an implant as disclosed herein, in particular an implant comprising a retaining element. Preferably, a delivery device as described herein is used to perform the method, preferably a delivery device comprising a retention element. The method comprises the steps of implanting the implant, and pulling at least one suture. The medical implant is released by pulling the suture.

In the following, the invention is described in detail with reference to the following figures, showing:

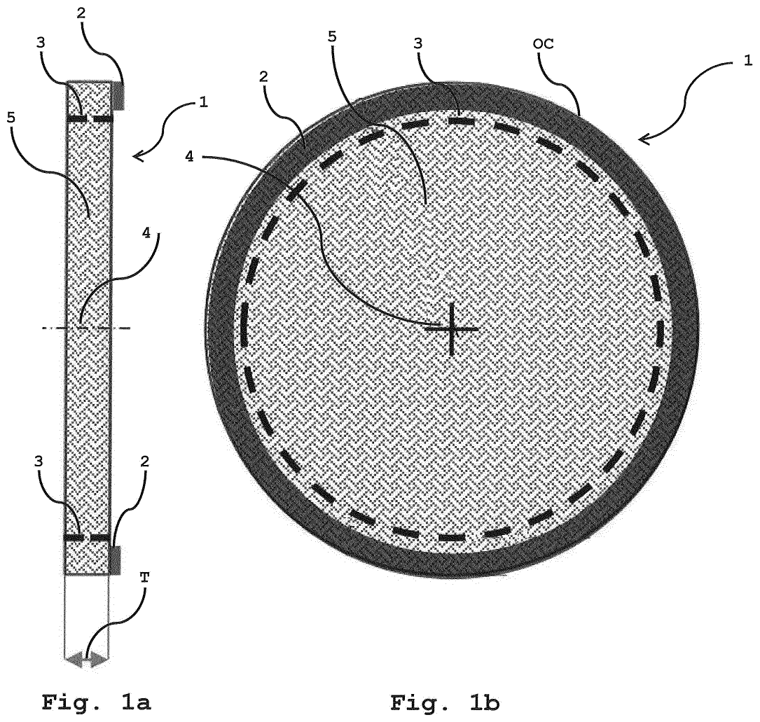

FIG. 1a-1b: an embodiment of a medical implant.

FIG. 2: an embodiment of a medical implant implanted into a patient.

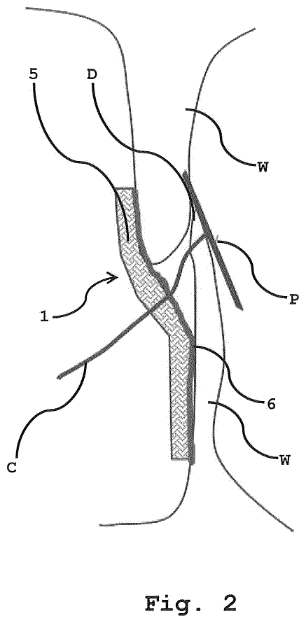

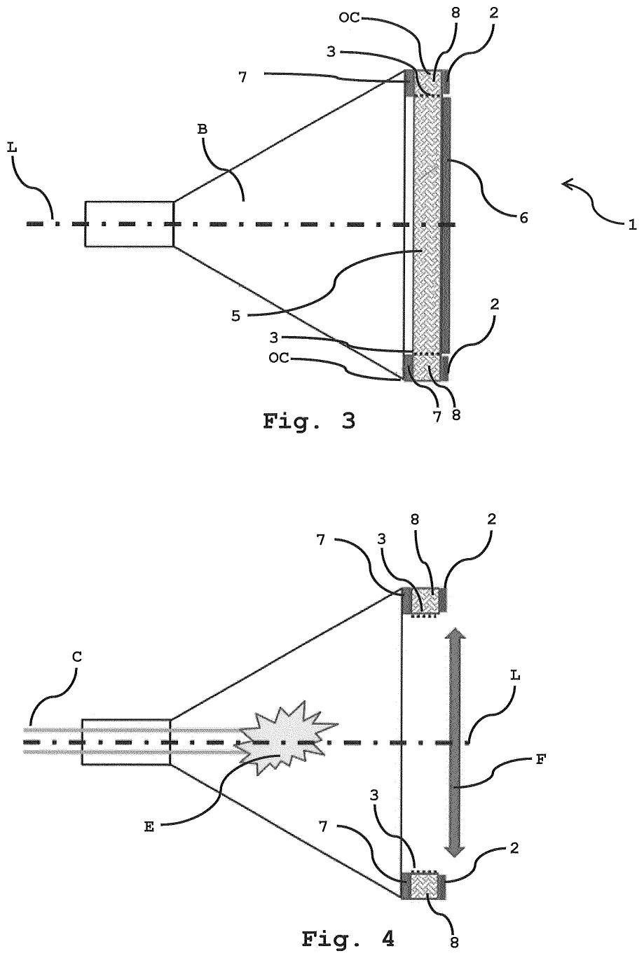

FIG. 3: an embodiment of a delivery device with a medical implant.

FIG. 4: an embodiment of a delivery device after release of a medical implant.

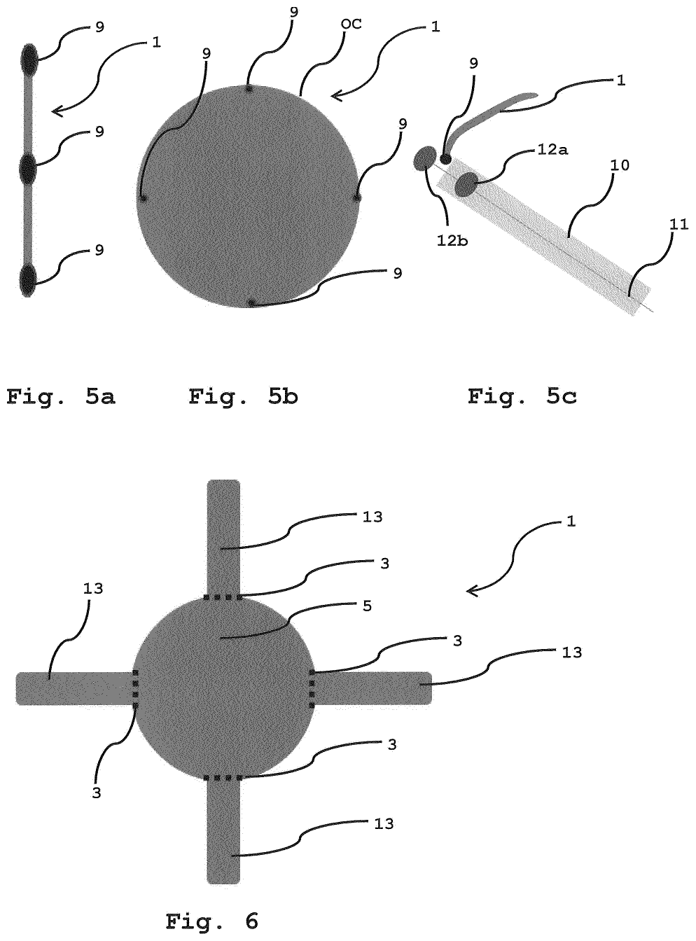

FIG. 5a-5c: an embodiment of a medical implant and schematically a release mechanism.

FIG. 6: an embodiment of a medical implant.

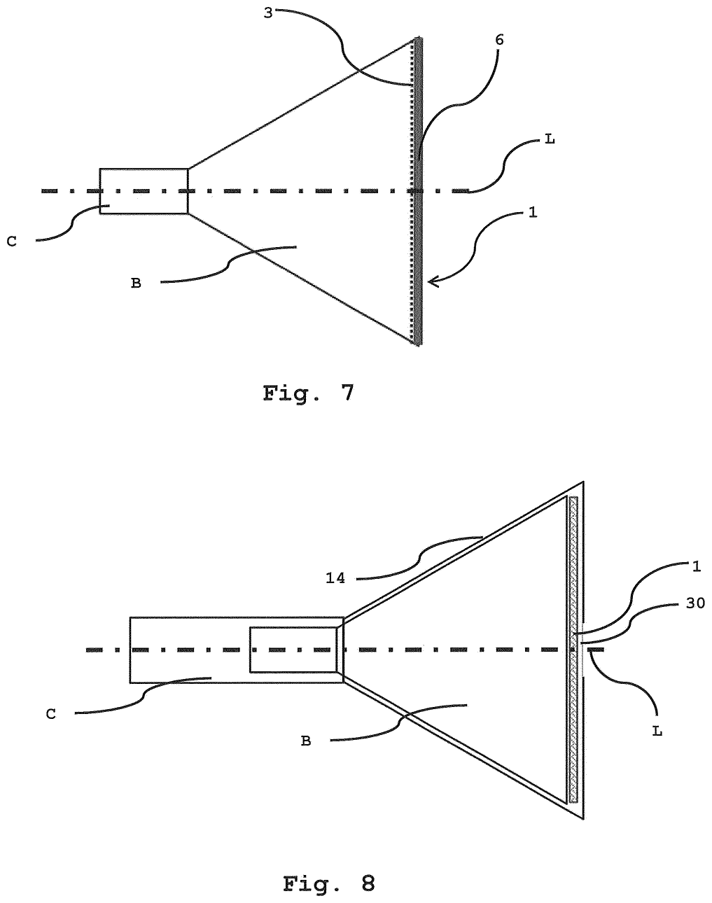

FIG. 7: an embodiment of a medical implant with a delivery device.

FIG. 8: an embodiment of a delivery device.

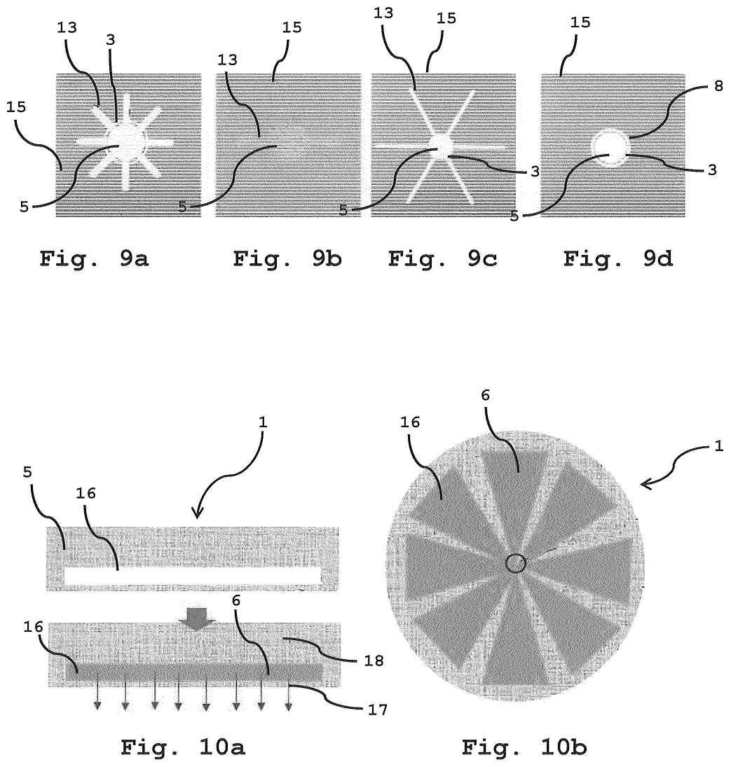

FIG. 9a-9d: different embodiments of a medical implant.

FIG. 10a-10b: an embodiment of a medical implant.

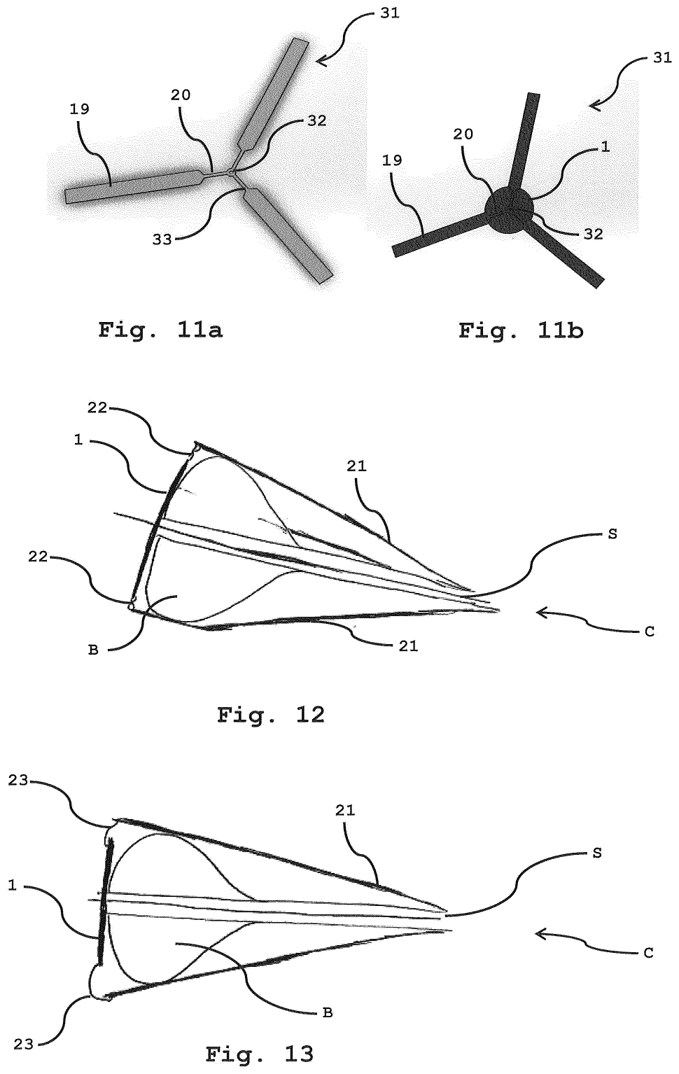

FIG. 11a-11b: an embodiment of a medical implant.

FIG. 12: an embodiment of a medical implant and schematically a release mechanism.

FIG. 13: an embodiment of a medical implant and schematically a release mechanism.

FIG. 14: an embodiment of a medical implant and schematically a release mechanism.

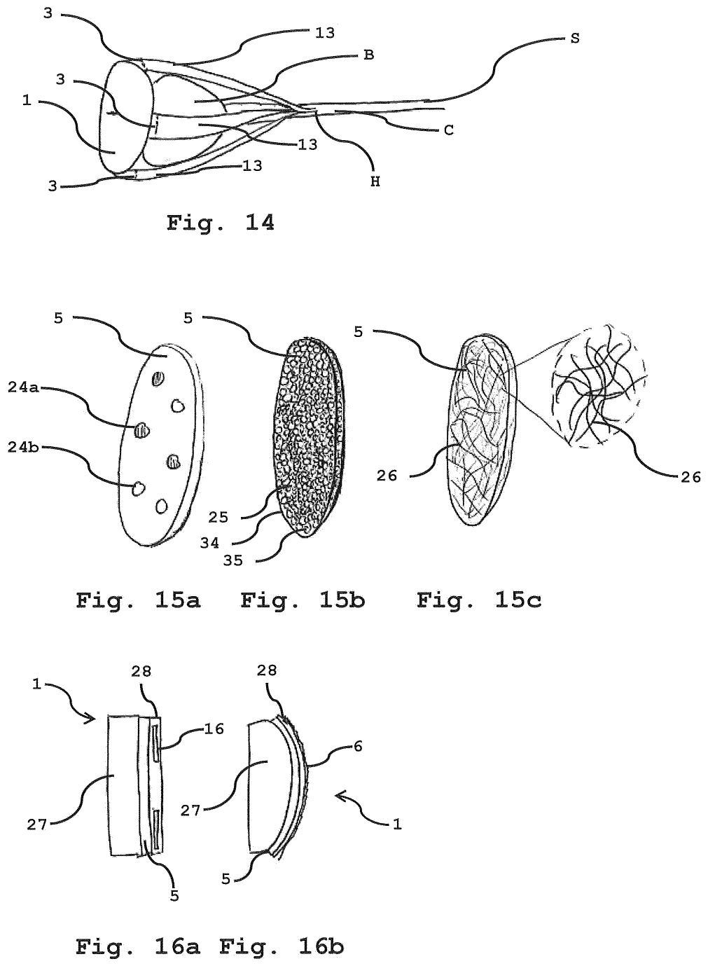

FIG. 15a-15c: schematically different embodiments of adhesive delivery.

FIG. 16a-16b: an embodiment of a medical implant.

FIG. 17: an embodiment of a medical implant implanted into a patient.

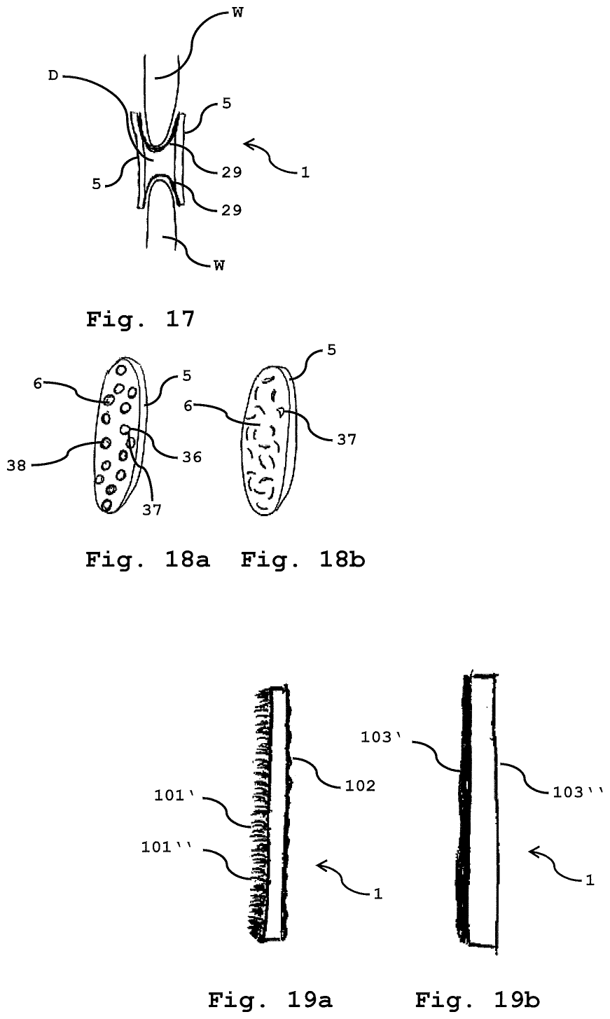

FIG. 18a-18b: a patch with capsules containing a filler material.

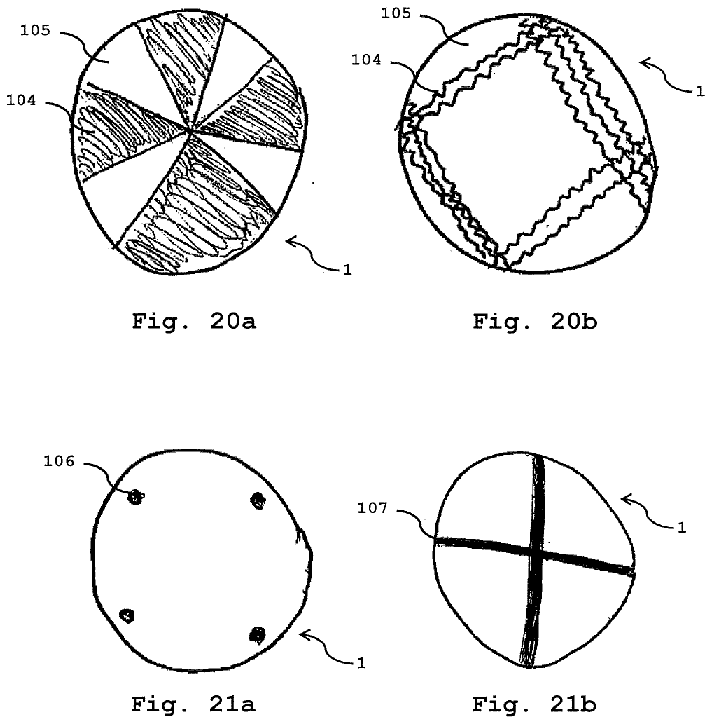

FIG. 19a-19b: patches with different first and second surfaces in a side view.

FIG. 20a-20b: patches with a patterned adhesive layer in a top view.

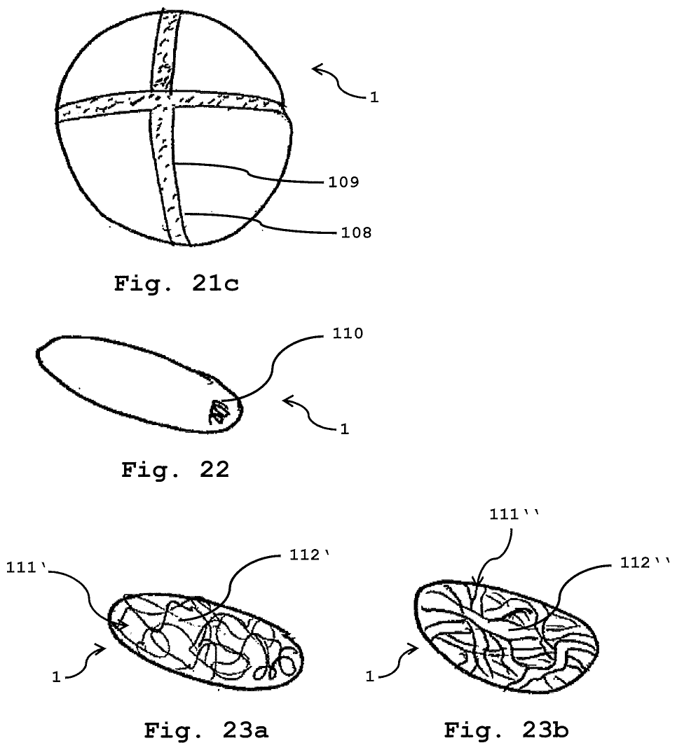

FIG. 21a-21c: patches with different embodiments of radio-opaque markers.

FIG. 22: a patch with a discrete marker.

FIG. 23a-23b: a patch with a dried adhesive before and after activation.

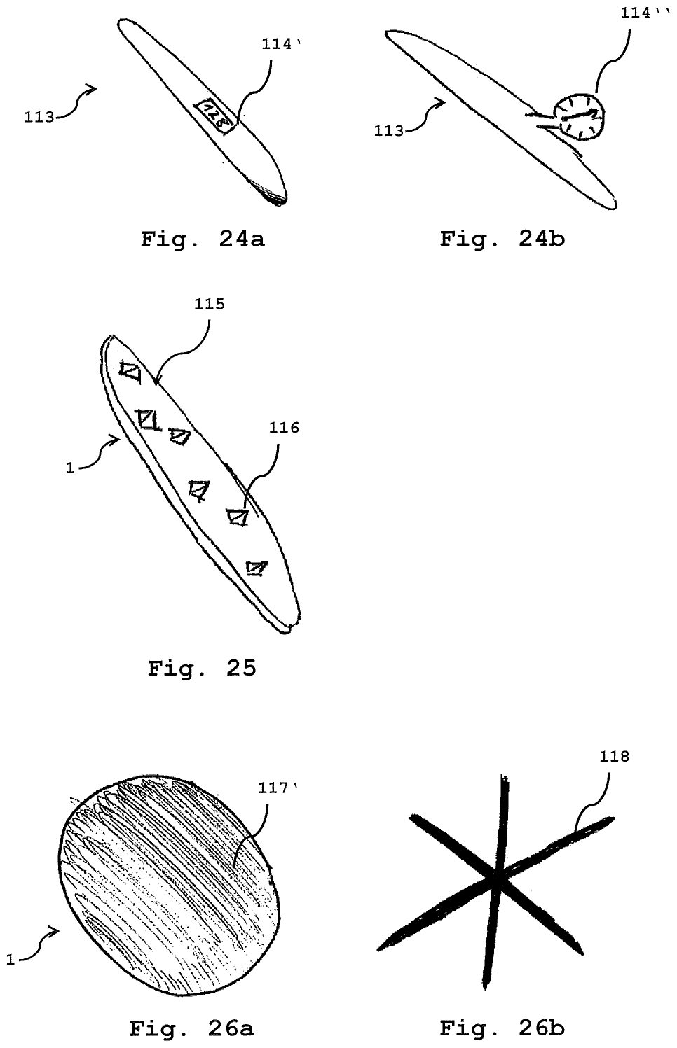

FIG. 24a-24b: two embodiments of a handle of a delivery device with a gauge.

FIG. 25: a patch with an adhesive having a three-dimensional pattern.

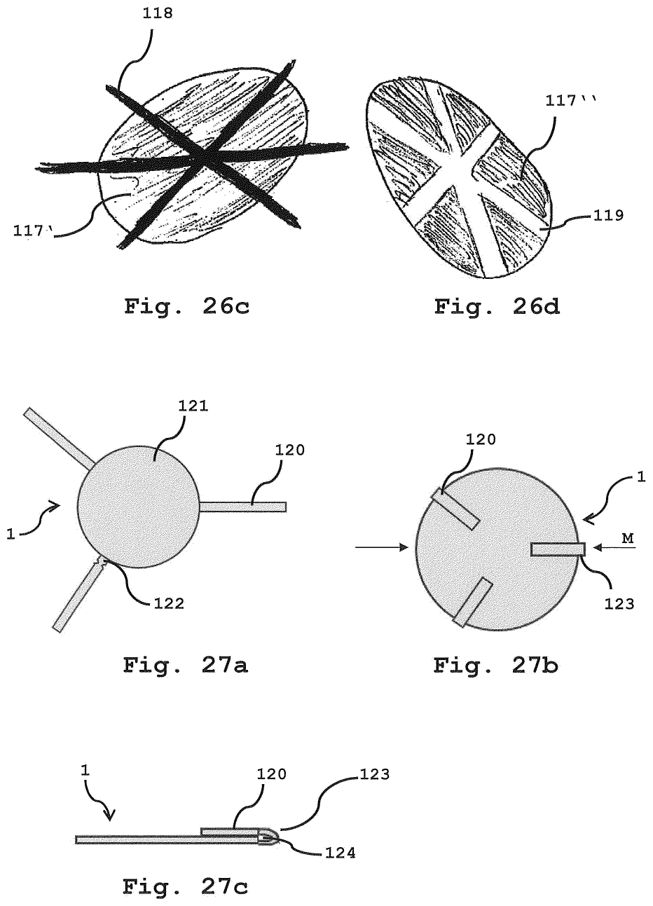

FIG. 26a-26d: schematically a method of patterning an adhesive layer on a patch.

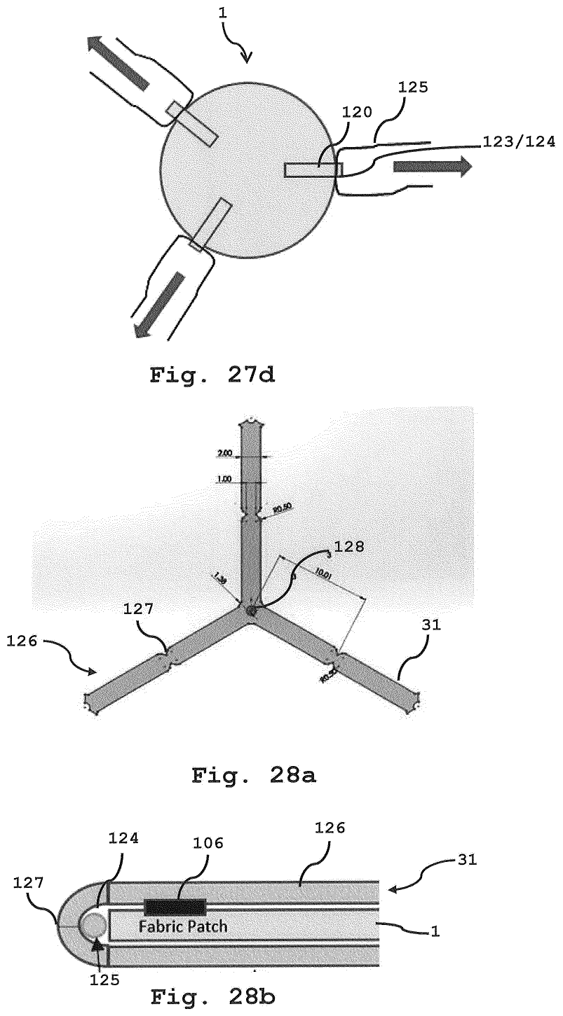

FIG. 27a-27d: schematically a method of producing a patch with a retaining element.

FIG. 28a-28b: schematically an embodiment of a backbone for a medical implant.

FIG. 29: a medical implant with a retaining element being attached to a retention element and

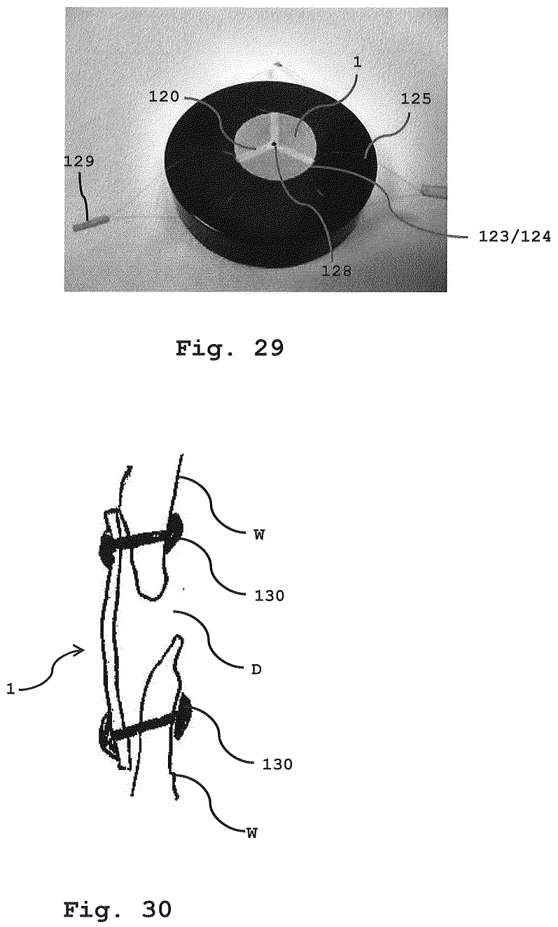

FIG. 30 schematically an implant attached to a tissue wall.

FIGS. 1a and 1b show an embodiment of a medical implant 1 according to the invention. The implant comprises a fabric patch 5 with a tear line 3. The fabric is a woven fabric of biocompatible fibers made of polyglycolic acid and is coated with a bioadhesive. Additionally or alternatively, the fibers may be made of another polymer such as PET.

FIG. 1a shows the medical implant 1 in a side view. Very well visible in the perspective is the silicone layer 2 that is only arranged on one side of the implant 1. This prevents adhesion between the medical implant and the tissue of the patient in the area that does not remain in the patient. Also visible in this perspective is the thickness T of the implant, which is 150 μm.

FIG. 1b shows a front view of the medical implant 1. The fabric is mechanically flexible and can adapt to the anatomy of the patient and the surface structure of the tissue at the implant site. The tear line 3 comprises a plurality of laser-cut parts along the circumference of the patch 5 that together form a circular pre-determined breaking line. The tear line is adapted to break upon radial stretch of the patch 5 and does not have any free fibers after tearing. Between the outer edge OC of the medical implant 1 and the tear line 3 is a layer silicone 2 as a non-adhesive material. The patch 5 further comprises a cross-shaped cut 4 in the in its center that is adapted such that a delivery device (not shown) can partially extend through it. The patch is adapted to degrade in the human body within six months. The woven structure facilitates tissue growth such that by the time the implant is degraded, it has been replaced with tissue. The implant has a diameter of 25 mm including the rim on the outer edge OC, and the patch has a diameter of 20 mm after tearing along the tear line 3.

FIG. 2 shows a medical implant 1 in the form of a patch 5 at an implant site. The implant site is a defect D in an atrial wall W of a patient's heart. Here, the medical implant 1 is shown during the implantation process. A delivery device C comprising a positioning device P partially extends through the patch 5 and cut in its center (not visible). The patch is coated with an adhesive composition 6, in this case GelMA. Alternatively, glutaraldehyde may be employed. This provides an effective attachment of the patch 5 to the atrial wall W.

FIG. 3 shows a similar medical implant 1 as shown in FIG. 2. Here, the implant is shown during the implantation process, but before detachment from the delivery device (not shown) comprising a balloon B. The medical implant comprises a patch 5 made of a knitted fabric that is attached to a balloon B by means of adhesive rims 7 one the balloon-side of the medical implant 1 and in between the outer edge OC of the medical implant 1 and its tear line. On the other side, the implant 1 is coated with an adhesive composition 6 within the area surround by the tear line 3. In between the tear line 3 and the outer edge OC, on the opposite site of the adhesive rims 7, is a PTFE coating that prevents wetting of the adhesive and thus adhesion. In the illustration shown here, the balloon B is partially inflated.

As shown in FIG. 4, further inflation applies a force F on the patch (not shown for clarity) and the tear lines 3 due to the extension, in a direction orthogonal to the longitudinal axis L of the delivery device, of the outer rims 8 of the medical implant that are attached to the balloon B through the adhesive rims 7. Thus, further inflation ruptures the tear line 3 and releases the patch 5. Here, the delivery device C is shown and is adapted to expose the patch to electromagnetic irradiation E.

FIGS. 5a-5c show schematically a medical implant 1 and a release mechanism for a medical implant 1. Here, the medical implant comprises a patch of spun fibers of polylactic acid. However, the person skilled in the art will of course understand that the release mechanism could be combined with any patch material or even any sort of medical implant. The implant 1 comprises beads 9 made of a polymeric material. Here, the beads are made of a biodegradable polymeric material that is adapted to degrade in the human body within typically two weeks. Of course, they could also be adapted to degrade faster or slower. They are attached to the edge OC of the medical implant 1.

FIG. 5a shows the medical implant 1 from the side. It is particularly well visible in this illustration that the beads 9 have a diameter that is larger than the thickness of the medical implant. Here, the beads 9 have a slightly elongated shape, but it would be possible to arrange spherical beads as well.

In FIG. 5b, the medical implant 1 is shown from a top perspective. It is well visible that the beads are considerably smaller than the medical implant. Typically, they have a diameter of 300 μm, but could also be up to 1 mm in diameter. Here, the implant 1 comprises four beads that are spaced equally around the circumference of the medical implant 1. It would of course be conceivable to arrange a higher or lower number of beads on the medical implant, and/or to space them unequally.

FIG. 5c shows schematically how an implant 1 as shown in FIGS. 5a and 5b can be released. The delivery device comprises at least one tube 10, typically one tube 10 per bead 9 attached to the medical implant 1. In said tube 10 a wire 11 with a lower holder 12a and an upper holder 12b. The holders 12a, 12b are adapted such that a bead 9 that is located between them in the tube 10 cannot pass the holders along the longitudinal direction of the tube. Thus, for implantation, the bead 9 is arranged in the tube 10 in between the upper holder 12b and the lower holder 12a. This enables the release mechanism shown in the illustration wherein the wire 11 is actuated such that the upper holder 12b is released from the tube 10. This clears the way for the bead 9 to also leave the tube 10, thus releasing the implant 1.

FIG. 6 shows another embodiment of a medical implant 1 and schematically a release mechanism. Here, the medical implant 1 comprises a patch 5 made of a biodegradable fabric. The fabric comprises fibers that are coated with an adhesive composition (not shown). The implant 1 comprises four extensions 13 that extend away from the implant 1. Each extension 13 is separated from the implant 1 by a tear line. Here, the extensions are made of the same material as the patch 5. This is particularly simple, but of course it would be possible to include other materials as well. The tear lines are arranged such that upon tearing of the tear lines, the patch 5 is substantially spherical and has a diameter of 20 mm.

FIG. 7 shows another embodiment of a medical implant 1. Here, a balloon B is attached to a delivery device (C). The balloon is made of implant-grade material, here from polyurethane. The balloon is coated with an adhesive composition 6 on a distal side. It further comprises a tear line 3 that is substantially circular and arranged in a plane that is substantially perpendicular to the longitudinal axis L of the delivery device C. Here, the tear line is formed as thinner wall part of the balloon B that creates a pre-determined breaking point. However, it is still sealed to allow for inflation of the balloon B. Inflation of the balloon B creates a mechanical stress in the balloon wall in a tangential direction. Due to the pre-determined breaking point, the balloon rupture along the tear line 3. The patch formed by the rupture adheres to the tissue by means of the adhesive composition 6. Thus, the medical implant was part of the balloon B during delivery.

FIG. 8 shows another embodiment of a delivery device C. Here, the delivery device C comprises a balloon B as described in other embodiments that can serve to deliver the medical implant 1. Of course, any medical implant 1 described herein can be combined with such a delivery device shown here. Here, the medical implant consists of a fabric patch with adhesive fibers. The delivery device comprises a second balloon 14 that forms an outer layer around the balloon B and the medical implant 1. It thus protects the patch and the adhesive from being in contact with tissue. Here, the outer balloon 14 comprises an opening 30 arranged approximately at the center of the medical implant 1. This provides an advantageous way to implant the medical implant 1, but is optional. This allows for a preliminary attachment to the tissue. For deployment, the outer balloon is retracted in a direction away from the implant 1 along the longitudinal axis L of the delivery device. This exposes the implant 1 to tissue and enables the adhesive composition to attach to the tissue.

FIGS. 9a-9d show different embodiments of fabric patches that are cut out from a fabric scaffold. Thus, the extensions 13 shown here consist of the same material as the patch 5. Throughout the FIGS. 9a-9d, only one reference sign is shown for certain identical features for clarity. The extensions 13 shown in these embodiments have a typical length of 15 mm and a width of 3.5 mm. Of course these values can be adapted to reach a particular mechanical strength or to adapt the patch to a particular delivery device.

FIG. 9a shows a patch with eight extensions 13 made from PET fabric 15. The extensions 13 are separated from the patch 5 by tear line 3 each. PET is a non-absorbable material. The shown embodiment is thus particularly advantageous if the replacement of the implant with tissue is not possible or undesired, for example due to insufficient stability of the newly formed tissue.

FIG. 9b shows an embodiment of a patch 5 with only two extensions 13. The fabric 15 consists of knitted poly(L-lactic acid) (PLLA). PLLA absorbs in in the human body within two years. Thus, the shown embodiment is particularly advantageous if cell-ingrowth is slow or support by the patch 5 for one to two years is desired.

FIG. 9c shows an embodiment of a patch 5 that is cut from a fabric 15 made of electrospun polycaprolactone (PCL). It comprises six extensions 13, each separated from the patch 5 by a tear line 3. PCL is particularly advantageous for electrospinning and thus provides an easy way to manufacture the fabric 15. It degrades in the human body within about six months and is thus the material of choice if relatively fast degradation is required or desired.

FIG. 9d shows another embodiment of a patch that made of the same electrospun PCL fabric 15 as shown in FIG. 9c. The patch 5 has a circular shape and a continuous circularly shaped tear line 3 around its circumference that forms an outer rim 8.

FIGS. 10a and 10b show another embodiment of a medical implant 1 in a cross-sectional view (FIG. 10a) and in a top view (FIG. 10b). For clarity, only one reference sign is shown for identical features. The medical implant 1 comprises an electrospun patch wherein the fibers are functionalized and have directionality. The implant 1 comprises a reservoir 16 that can be filled with an adhesive composition. A region 18 of the patch 5 can be functionalized such as to have a lower permeability. Here, this prevents an adhesive disposed in the reservoir 16 from permeating through the patch and be released this side of the medical implant. Instead, the shown embodiment comprises microchannels 17 integrated by selective laser welding. These microchannels 17 are in fluid connection with the outer surface of the medical implant 1 and the reservoir 17. The adhesive can thus be released in a directional manner by means of the microchannels.

FIGS. 11a and 11b show an embodiment of a spine structure 31. FIG. 11a shows a spine structure 31 by itself. The shown spine structure is made of a polymer, similar or different from the patch. It comprises three elongated structures 19, 20. This is typically the most advantageous arrangement in that it provides sufficient mechanical stability to the implant. However, it would also be possible to adapt a spine structure with several additional elongated structures, or with only one or two of them, if necessary. The elongated structures comprise an inner part 20 and tear arms 19 that a separated by a predetermined breaking point 33. The elasticity of the tear arms is generally lower than that of the inner part. The inner part 20 is designed to be arranged in an area close to a medical implant 1 and remain in the patient upon implantation. Thus, the length of one arm of the inner part 20 is approximately half a diameter of the implant, typically around 10 mm. Of course, the size of the spine structure 31 as a whole and of the inner part 20 can be adapted to a specific implant and thus be larger or smaller. The inner part gives the implant mechanical stability during delivery and implantation, and also provides additional support after implantation. The spine structure 31 further comprises a round-shaped hole 32 in the middle of structure 31. This enables a delivery device and/or positioning device (both not shown) to extend through the spine structure 31 and be retracted through it again. Furthermore, the less elastic tear arms 19 are can be attached to a delivery device. Breaking at the predetermined breaking point allows for the release of the implant 1. A spine structure 31 as shown here provides a particularly advantageous way of decoupling the force needed to release the patch from the mechanical properties of the medical implant 1.

FIG. 11b shows a spine structure with the same features as shown in FIG. 11a but in combination with a medical implant 1.

FIG. 12 shows a delivery device C for a medical implant 1 and schematically a release mechanism. The delivery device C comprises an inflatable or expandable structure, such as a balloon B and outer struts 21 to hold and release the medical implant 1. Here, the medical implant comprises suture with notches 22 that are held by the outer struts 21. The mechanism of holding and releasing the notches substantially corresponds the ball release schematically shown FIG. 5c, wherein the notches 22 have the technical effect of the balls shown in FIG. 5c. The balloon B can thus be used to exert a pressure on the medical implant, but is not necessary to release the implant 1 from the delivery device C.

FIG. 13 shows a similar delivery device C as shown in FIG. 12. However, the outer struts 21 here are connected to the medical implant 1 through sutures 23. The sutures are fixedly connected to the outer struts 21 that do not comprise a mechanism to release the sutures 23. Instead, the balloon B, when inflated, pushes the outer struts 21 away from the medical implant, thus rupturing the connection and releasing the implant 1.

FIG. 14 shows schematically a delivery device C with a medical implant 1 similar to the one shown in FIG. 6. The implant 1 comprises elongated flaps 3 that are connected to the implant 1 through tear lines 3. The shown embodiment of the implant 1 has four such flaps 13, but could also be adapted to have smaller or larger number of flaps. The delivery device comprises a balloon B. Upon inflation, the balloon exerts a force on the tear lines 3, causing them to rupture and release the implant. The flaps 13 can then be retracted together with the delivery device C, while the medical implant 1 remains in the patient. Although not shown here, the embodiment illustrated is well suited to be combined with spine structure as shown in FIGS. 11a and 11b.

FIGS. 15a-15c show schematically different embodiments of patches 5 comprising an adhesive composition.

FIG. 15a shows a patch 5 comprising two different types of cavities 24a, 24b. The cavities 24a, 24b are spherical and have a diameter of approximately 1 mm. The patch has a diameter of 20 mm and consists of poly(lactic acid-co-glycolic acid). Other absorbable materials such as PLA-GA, PLGA, PCL, PU could also be used. Alternatively, non-absorbable materials such as PET, PE, and/or PP may be used. The cavities 24a, 24b contain two different components, a resin and a hardener, of an adhesive composition. The resin and the hardener are physically separated from one another and become curable upon mixing. Thus, the adhesive composition is not curable in the shown state where the two components are separated. However, cavities 24a, 24b in the shown embodiment are adapted to burst upon application of a mechanical pressure, for example exerted by an inflatable balloon. The burst of the cavities causes the release of both components of the adhesive composition, rendering it curable. Typical adhesives might be GelMA (Metacrylated Gelatin), CollMA (methacrylated Collagen) or MeTro (methacrylated Tropoelastin).

FIG. 15b shows a patch that comprises a foam 25. The pores 35 of the foam are surrounded by walls 34 made of a hydrogel. The hydrogel is adapted to degrade in the human body within 24 months. The pores 35 are filled with an adhesive composition. The patch is adapted to release the adhesive composition from the pores 35 upon a mechanical deformation. The adhesive composition here is curable by exposure to electromagnetic radiation. It will be understood by the person skilled in the art that any adhesive composition could be combined with the shown patch, in particular any curing mechanism. The pores 35 are adapted in their size to facilitate cell in-growth such that after release of the adhesive composition, the empty pores can serve as a scaffold for tissue growth. The biodegradation of the patch 5 is adapted such that the patch degrades after the formation of new tissue.

FIG. 15c shows yet another embodiment of a patch 5. The shown patch is made of electrospun fibers 26 of polycaprolactone. The fibers have a diameter of approximately 3 μm and a length of several 100 μm. The fibers are coated with methacrylated gelatin as an adhesive composition. This patch can thus easily be attached to tissue and is particularly easy to fabricate by electrospinning. It will of course be understood by the person skilled in the art that the fibers could also consist of an adhesive composition instead of being coated by it. Similarly, although polycaprolactone is particularly advantageous for electrospinning, the fibers could be made of another material.

FIGS. 16a-16b show an embodiment of medical implant 1 in a lateral cross-section. The implant 1 comprises an inflation reservoir 27, a patch 5 and a separate layer 28 comprising reservoirs 16 for containing an adhesive. The shown embodiment is similar to the medical implant shown in FIGS. 10a and 10b.

FIG. 16a shows the medical implant 1 in a first state. The inflation reservoir is empty. The reservoirs 16 for comprising an adhesive are filled with adhesive. Typically the adhesive can be a poly(acrylic) acid which uses an acrylate/methacrylate/amuse crosslinker.

The patch 5 is made of electrospun fibers of Dacron and is not biodegradable. However, it would of course be possible to adapt the patch 5 to be biodegradable as well. The layer 28 comprising the reservoirs 16 for the adhesive is of solid Dacron.

FIG. 16b shows the medical implant 1 in a second state wherein the inflation reservoir 27 is inflated. The inflation of the inflation reservoir 27 applies a mechanical pressure to the reservoirs 16 containing the adhesive which is subsequently squeezed out, forming a layer of adhesive 6 on one side of the implant 1. Here, the patch 5 is adapted to not be penetrable by the adhesive composition, thus leading to a selective release of the adhesive composition on the other side of the implant. The inflation reservoir is adapted to be removed after inflation. However, it would also be conceivable to form it from an implant grade material that remains in the patient.

FIG. 17 shows another embodiment of the medical implant according to the invention in an implanted state closing a defect D in a heart wall W. The implant 1 comprises a support structure 29 that extends around a circumference of the defect D. The support structure is made of a shape memory polymer that provides self-expanding at the implant site. Upon expansion, it is engaged in the defect D. Two patches 5 are attached to the support structure 29. The patches in the shown embodiment comprise electrospun Dacron fibers coated with a methacrylated collagen that swells upon exposure to humidity in the body. However, any patches as described herein can be used, of course.

It will of course be understood by the person skilled in the art that the embodiments described herein are examples are not restrictive to the scope of the invention. In particular, the different features described herein may be freely combined with other features and/or used without certain features.

FIGS. 18a and 18b show an embodiment of a patch 5 comprising capsules 36 with a filler material 37 in the capsule wall 38.

FIG. 18a shows the patch in a first state. The capsules 36 are spherically shaped and have a diameter of approximately 0.5-2 mm and are evenly distributed in the patch 5. An adhesive composition 6 comprising methacryloyl-substituted tropoelastin is contained in the inside of the capsules 36. Here, the capsules 36 are adapted to break open due to an osmotic pressure. For example, exposure to blood or another liquid causes swelling of the capsules 36. The resulting pressure increase then causes bursting of the capsules 36.

FIG. 18b shows the patch 5 in a second state after rupture of the capsules 36. The adhesive composition 6 is evenly distributed over the surface of the patch 5. The filler material 37 remains in the adhesive composition 6 and provides additional mechanical strengths to the adhesive layer.

FIG. 19a shows a patch 1 in a side view. The patch 1 has a first surface 101′ and a second surface 102. The first surface 101′ is configured as a velour-like surface having shorts strands of fabric 101″ extending away from the first surface 101′. The second surface 102 comprises an adhesive layer. The medical implant 1 is made of a polyurethane, and the velour-like surface 101′ comprises polyurethane fibers. In the shown configuration, the velour-like surface 101′ enhances cell ingrowth and thus tissue overgrowth, while the second surface 102 provides adhesion to tissue.

FIG. 19b shows a similar embodiment as shown in FIG. 19a. The medical implant 1 comprises a first surface 103′ and a second surface 103″. The first surface 103′ has a lower permeability for an adhesive composition (not shown) than the first surface 103′. In the present case, this is achieved by a thicker layer of porous material. The implant comprises the adhesive composition and can release it by means of a sponge-like mechanism when mechanical pressure is applied. The adhesive composition preferentially permeates the second surface 103″ and thus, the second surface 103″ provides more adhesion as compared to the first surface 103′, when activated.

FIG. 20a shows a patch 1 with an adhesive layer 104 that has a patterned structure. The pattern is configured as five sectors of a circular shape that forms the patch 1. Consequently, there are four intermediate sectors 105 that do not comprise an adhesive layer. The adhesive layer 104 was printed via inkjet printing and is based on a mixture of porcine and fish GelMA. Alternatively, extrusion printing may also be employed. Presently, the pattern is two-dimensional. The adhesive layer is thus substantially flat and the sectors 104,105 of the patch 1 differ in whether or not they have an adhesive layer, but not in the thickness of said adhesive layer.

FIG. 20b shows a medical implant similar to the one shown in FIG. 20a. The patch 1 comprises an inkjet-printed pattern of adhesive 104. The adhesive pattern is two-dimensional and is arranged substantially at the circumference of the patch 1. The adhesive pattern comprises several curved lines.

It will be understood that any particular pattern of adhesive may be arranged on a patch, in particular if the adhesive is inkjet printed. Alternatively, extrusion printing may also be employed.

FIG. 21a shows an embodiment of a patch 1 with a radio-opaque element 106. The radio-opaque element 106 consists of four beads comprising barium sulphate arranged at a circumferential area of the patch 1 and spread substantially equally along in the direction of the circumference of the patch 1.

FIG. 21b shows an alternative embodiment of a patch 1 having a radio-opaque element 107. The radio-opaque element 107 consists of a cross-shaped metallic spine structure.

FIG. 21c shows an alternative embodiment of a radio-opaque element 108,109 on a patch 1. A spine structure 108 made of a polyurethane comprises small capsules 109 filled with iodine. The iodine provides radio opacity.

FIG. 22 shows a patch 1 with a discrete marker 110. The discrete marker 110 is configured as a spring-like element. The discrete element 110 consists of titanium and is thus also radio-opaque and echo-opaque. However, it would be possible to alternatively configure the discrete marker 110 to not be radio-opaque and/or echo-opaque. The discrete marker is deformable by pressure and thus provides information on the pressure acting on the patch 1 at its location. Presently, the pressure can be read by measuring the extension of the marker 110 along a longitudinal axis (perpendicular to the surface of the patch 1), for example via radiography.

FIG. 23a shows a medical implant 1 with a dried adhesive 111′. The dried adhesive comprises fibers 112 of porcine GelMA spun from an aqueous solution and then dried. Any gelatin may also be employed as in addition or as an alternative to porcine GelMA.

FIG. 23b shows the medical implant 1 of FIG. 23a after exposure to an aqueous liquid. The adhesive composition 111″ is swollen from water incorporation and the fibers 112″ are thus larger in diameter as compared to the dried fibers 111′,112′. In the swollen state, the fibers 112″ exhibit an adhesive force to human tissue.

FIG. 24a shows a handle 113 for a delivery device. The handle 113 comprises a digital gauge 114′ that is adapted to show a numerical value representing an adhesive force between an implant as shown herein and human tissue (not shown) to which it is attacked. The gauge 114′ is attached to the implant at a distal end and measures the adhesive force.

FIG. 24b shows an alternative embodiment of a handle 113. An analog gauge 114″ provides a qualitative measure (for example high, medium, low) of an adhesive force between an implant as shown herein and human tissue (not shown) to which it is attached.

It will be understood that a digital gauge may also be used to show a qualitative measure and/or an analog gauge may be used to show a numerical value.

FIG. 25 shows an embodiment of a medical implant 1 with a three-dimensionally patterned adhesive 115. The adhesive is formed as pyramids 116 evenly spaced over one surface of the implant. The medical implant is configured as a patch made of pericardium. The adhesive 115 consists of pyramids 116 of fish GelMA.

FIGS. 26a-26d schematically show a method of patterning an adhesive composition on a medical implant 1.

FIG. 26a shows a medical implant 1 made of polyurethane with a smooth, homogeneous layer 117′ of adhesive. The adhesive is based on bovine GelMA.

FIG. 26b shows a stamp-like element 118. The stamp-like element has a shape representing a negative of the desired adhesive pattern on the medical implant 1. The stamp-like element 118 is made of a metallic material with a PTFE coating. Thus, the stamp-like element 118 does not adhere to the adhesive layer 117′ and can be removed easily from the adhesive bearing surface.

FIG. 26c shows the stamp-like element 118 being pressed on the adhesive layer 117′ of the medical implant 1. The stamp-like element 118 pushes the adhesive laterally away.

FIG. 26d shows the medical implant 1 after removal of the stamp-like element. The adhesive layer 117″ has a pattern that substantially corresponds to a negative shape of the stamp-like element. The medical implant 1 hence comprises areas 119 that are not covered by an adhesive layer. The aggregate of the area 119 substantially corresponds to the shape of the stamp-like element.

Any of the implants and adhesives disclosed herein are suitable to be patterned with the method shown in FIGS. 26a-26d. It would also be possible to pattern a three-dimensional pattern using the method of FIGS. 26a-26b.

FIGS. 27a-27d schematically show a method to produce a medical implant 1 comprising a retaining element for holding suture(s).

FIG. 27a shows a first step of the method. The medical implant 1 is arranged with three radially extending flaps 120. In the present case, the extending flaps 120 are made of polyurethane and separately attached to a patch 121 made of pericardium. One of the three extending flaps 120 comprises an indentation 122 that functions as a pre-determined breaking point to reduce the necessary force to break the extending flap and/or to control where breaking occurs. It would alternatively be possible to have any number of extending flaps 120 with or without indentation.

FIG. 27b shows the medical implant 1 of FIG. 27a, wherein the extending flaps have been folded towards the center of the medical implant 1. After folding, the medical implant 1 has a substantially round shape. Around an area of the fold 123 a passage is formed (not visible, see FIG. 27c).

FIG. 27c shows a cross-section of the medical implant 1 of FIG. 27b along the plane M. In the area of the fold 123, a passage 124 is formed that can be used, for example, to hold a suture (not shown).

FIG. 27d shows schematically an embodiment of a medical implant 1 as shown in FIGS. 27a-27c. The implant 1 is held at the fold 123 in the passage 124 by sutures 125. Pulling of the sutures 125 releases the implant 1 by tearing the extending flaps 120 at the area of the fold 123.

FIG. 28a shows an embodiment of a spine structure 31 made of a polyurethane. The spine structure 31 in generally suitable to be combined with any of the disclosed medical implants. The spine structure 31 comprises three arms 126. Each arm has a thickness of 2 mm and further comprises an indentation 127, where the arm 126 has a reduced thickness of 1 mm. The indentation 127 is placed at a distance of 10 mm from the center 128 of the spine structure 31. Thus, when arranged on a medical implant, the indentation is typically located at the circumference of the implant. The spine structure is also configured to extend beyond the circumference of an implant in this case, and is therefore particularly suited to produce an implant as shown in FIGS. 27a-27c.

FIG. 28b shows a cross-section of a spine structure similar to the one shown in FIG. 28a. The arm 126 is folded around the indentation 127. In the shown embodiment, the spine structure 31 is attached to a medical implant 1 configured as a fabric patch.

The arm 126 was bonded onto the fabric of the implant 1 by heat bonding wherein the polyurethane was partially molten and diffused into the fabric, thus providing adhesion. The folded arm 126 forms a passage 124 through which a suture is passed. The folded arm 126 thus forms a retaining element and is held by a suture 125. The suture is adapted in its mechanical strength (i.e. thickness and material choice) such that pulling of the suture may tear the arm 126 of the spine structure 31 at the indentation 127. The implant further comprises a radio-opaque element 106, configured as a platinum particle, held on the implant 1 by the folded arm 126. As an alternative to platinum, iridium is also suited as a material for a radio-opaque marker. Additionally or alternatively, the spine structure 31 could be made from a polymer filled with radiopaque agents, such as BaSO4.

FIG. 29 shows an implant 1 according to the invention. The implant 1 is similar to the one shown in FIG. 27d. The implant comprises extending arms 120 cut from the same base sheet material as the body of the implant 1. The arms 120 were folded onto the patch 1, thus leaving a small passage 124 at a circumferential area of the patch 1 to allow a suture 125 to pass through. The sutures 125 can be mounted to a delivery system via retention elements 129. Any delivery system as shown in FIGS. 12-14 is suitable to be combined with the retention elements 129. Pulling the sutures backwards causes the sutures 129 to cut through the polymeric sheet and thus releases the implant 1. The required force for the cut can be controlled/adjusted by cutting small indentations into the arm 120, as shown in FIG. 27a. The implant further comprises a hole in a center region 128 that enables additional holding with a delivery instrument, for example for easier centering of the implant 1 at an implantation site.

FIG. 30 shows schematically an implant 1 attached to a tissue wall W such as to close a defect D. The implant 1 is attached to the tissue wall W by means of two rivets 130. The rivets consist of a biodegradable material and degrade in a human body within a year. Thus, the rivets 130 provide temporary attachment while until, for example, a sufficient adhesive force has formed and/or tissue has formed on the implant 1.

1.-74. (canceled)

75. A medical implant comprising an adhesive composition, the medical implant comprises two states, wherein the medical implant in its first state can be deployed to an implant site while the adhesive composition is inactive, and brought into the second state, by an activation mechanism, the adhesive composition, in said second state, is curable by a curing mechanism.

76. The medical implant according to claim 75, comprising a radiopaque element.

77. The medical implant according to claim 76, comprising a support structure.

78. The medical implant according to claim 76, wherein the radio-opaque element is arranged within or formed by the adhesive composition.

79. The medical implant according to claim 76, wherein the radio-opaque element comprises at least one of barium sulfate and iodine.

80. The medical implant according to claim 75, wherein the medical implant has a generally flat shape with a first and a second surface, wherein the first and the second surfaces have substantially opposite orientations, and wherein at least one property of the first surface is different from a corresponding property of the second surface.

81. The medical implant according to claim 80, wherein the first surface is adapted to enhance cell ingrowth.

82. The medical implant according to claim 80, wherein the second surface is adapted to provide adhesion to biological tissue.

83. The medical implant according to claim 75, wherein at least one surface of the implant, comprises a velour-like surface.

84. The medical implant according to claim 75, wherein the adhesive composition is arranged on the medical implant in a pattern.

85. The medical implant according to claim 75, wherein the adhesive composition comprises GelMA.

86. The medical implant according to claim 85, wherein the GelMA is of animal origin selected from Fish GelMA or porcine GelMA.

87. The medical implant according to claim 85, wherein the GelMA is formed by a mixture of at least two GelMAs of animal origin.

88. The medical implant according to claim 85, wherein the adhesive composition further comprises a photoinitiator.

89. The medical implant according to claim 85, wherein the GelMA is cross-linkable by X-ray radiation.

90. The medical implant according to claim 75, comprising at least retaining element for retaining a suture at an outer circumference of the implant, and a suture that is arranged within said loop.

91. The medical implant according to claim 75, wherein the adhesive composition comprises a dried adhesive composition.

92. A medical implant according to claim 91, wherein the dried adhesive composition is activatable by exposure to a liquid.

93. A method for deploying a medical implant that comprises an adhesive, comprising the steps
deploying the medical implant to a first site in a first state,
bringing the medical implant into a second state by means of an activation mechanism,
where the adhesive is cured by means of a curing mechanism.

94. The method according to claim 93, comprising the step of increasing the temperature to bring the medical implant into the second state.

95. The method according to claim 94, wherein the temperature increase is at least partially provided by an external heat source.

96. The method according to claim 93, wherein the temperature increase is at least partially provided by a patient's body heat.

97. The method according to claim 93, wherein the temperature increase is at least partially provided by electromagnetic radiation.

98. The method according to claim 93, comprising the step of applying a pressure increase to bring the implant into the second state.

99. The method according to claim 98, wherein the pressure increase is at least partially caused by osmotic pressure.

100. The method according to claim 93, comprising the step of exposing the implant to humidity to bring it into the second state.

101. The method according to claim 93, comprising the step of spontaneous mixing of two components in the second state, which causes curing of the adhesive.

102. The method according to claim 93, comprising the step of exposing the implant to electromagnetic radiation in the second state, which causes curing of the adhesive.

103. The method according to claim 93, comprising the step of disposing a liquid to bring the medical implant into the second state.

104. The medical implant according to claim 75, comprising a biodegradable material that is adapted to lose its mechanical strength in a human body within six to 36 months.

105. The medical implant according to any claim 75, comprising a spine structure.

106. A method of producing a medical implant, wherein an adhesive composition is at least partially liquid and is arranged on at least one surface of the medical implant, further comprising a step of drying the adhesive composition.

107. A method of producing a medical implant, comprising the steps of arranging at least one extension element at an outer circumference of the medical implant, configure the at least one extension element to form a retaining element, bonding an end of the extension element such as to fix the extension element it a configuration comprising a retaining element.
